(19) United States
(12) Patent Application Publication
(10) Pub. No.: US 2026/0363899 A1
(43) Pub. Date: Nov. 19, 2026

(54) METHOD AND APPARATUS FOR RESTRAINT ANCHOR

(71) Applicant: Andrew NUZER, Point Pleasant, NJ (US)

(72) Inventor: Andrew NUZER, Point Pleasant, NJ (US)

(21) Appl. No.: 19/351,984

(22) Filed: Oct. 7, 2025

Related U.S. Application Data
(63) Continuation-in-part of application No. 18/402,625, filed on Jan. 2, 2024, now Pat. No. 12,502,303.
Application No. 18/402,625 is a continuation of application No. 17/498,691, filed on Oct. 11, 2021, now Pat. No. 11,857,452.
Application No. 17/498,691 is a continuation of application No. 16/849,925, filed on Apr. 15, 2020, now Pat. No. 11,141,305.
(60) Provisional application No. 62/834,355, filed on Apr. 15, 2019.
(60) Provisional application No. 62/874,615, filed on Jul. 16, 2019.
(60) Provisional application No. 63/815,974, filed on Jun. 2, 2025.

(51) Int. Cl. A61F 5/37; A44B 11/25; A47B 91/08

(52) U.S. Cl. CPC A61F 5/3776; A44B 11/2588; A47B 91/08

(57) ABSTRACT

An anti-ligature restraint device is provided. The device includes a tub secured to a mounting location. A sealing element is inserted into the tub. The sealing element includes a restraint element slot to which a restraint is secured when the device is in an open position. The sealing element is disposed entirely with the tub in the closed position. A spring within the tub provides resistance to the sealing element as it is pushed into the tub and rotated to secure the sealing element within the tub. A lid lock secures the sealing element in the closed position. The device may be used in the concrete floor of a hospital room. In the open position, the device secures patient restraints. In the closed position, the device is flush with its mounting surface and locked, thereby preventing patient tampering or access to harmful elements thereof.

### CROSS-REFERENCE TO RELATED APPLICATIONS

This application is a continuation-in-part of non-provisional utility U.S. patent application Ser. No. 18/402,625, titled METHOD AND APPARATUS FOR RESTRAINT ANCHOR” filed on Jan. 2, 2024; which in turn is a continuation of non-provisional utility U.S. patent application Ser. No. 17/498,691, titled METHOD AND APPARATUS FOR RESTRAINT ANCHOR” filed on Oct. 11, 2021, now U.S. Pat. No. 11,857,452 issued Jan. 2, 2024; which in turn is a continuation of non-provisional utility U.S. patent application Ser. No. 16/849,925, titled METHOD AND APPARATUS FOR RESTRAINT ANCHOR” filed on Apr. 15, 2020, now U.S. Pat. No. 11,141,305 issued Oct. 12, 2021; which in turn is a non-provisional utility U.S. Patent Application that claims priority to two U.S. Provisional Patent Applications: Ser. No. 62/834,355, titled “METHOD AND APPRARTUS FOR RESTRAINT ANCHOR” filed on Apr. 15, 2019 and Ser. No. 62/874,615, titled “METHOD AND APPRARTUS FOR RESTRAINT ANCHOR” filed on Jul. 16, 2019.

This application also claims priority to U.S. Provisional Patent Application Ser. No. 63/815,974, titled “METHOD AND APPRARTUS FOR RESTRAINT ANCHOR” filed on Jun. 2, 2025.

The entire contents of each and every one of these patent applications is incorporated by reference as if set forth herein in their entirety into the present patent application as if written into the same.

### COPYRIGHT & TRADEMARK/TRADENAME NOTICES

A portion of the disclosure of this patent document may contain material, which is subject to copyright protection. The owner has no objection to the facsimile reproduction by any one of the patent document or the patent disclosure, as it appears in the Patent and Trademark Office patent file or records, but otherwise reserves all copyrights whatsoever.

### BACKGROUND OF THE INVENTION

### 1. Field of the Invention

The invention relates to the provision of an anti-ligature restraint anchor. In particular, secure hospital settings such as those for the mentally and emotionally challenged, require certain secure restraint mechanisms. In a first, operable, position the restraint mechanism provides a patient restraint securing location at which point a secure strap or other restraint may be secured. In a second, safe and secure, position the restraint mechanism is “closed” or “inaccessible” such that the hospital patient has no useful access to the mechanism. In this second position, the patient cannot use the device to cause harm to themselves. Such a device provides proper and secure restraint in the first position when the patient requires restraint, while providing a safe, anti-ligature enclosure in the second position when the patient is free to move about and has potential access to the restraint device.

As evident to those skilled in the art, the use of the devices disclosed in the present invention may also be used in other environments to provide similar functions. For example, any location requiring secure holding locations are candidates for the use of the present invention including but not limited to jail cells, prisons, airports, pet or livestock staging locations etc.

### 2. Description of Related Art

Prior art devices are available in the anti-ligature space and include institutional apparatus for all aspects of protected environments. Various shower installations, shower heads, door handles, beds, door frames and other similar apparatus are available for safe use within such protected environments. However, no devices are available that provide an intentional ligature point such that the intentional ligature is capable of being conveniently and safely hidden without removing it from the protected environment when not in use.

US Pat. Pub. No. 2011/0198458A1 (Karl, et al.) discloses a tamper resistant glide for furniture. The tamper resistant glide assembly comprises a foot, a stem and an insert connected to a leg of the furniture. The foot has a having a top and a bottom; and the bottom is adapted to engage a floor surface. A stem mount on the top of the foot provides an adjustable attachment to a first end of the stem. The stem has a second end spaced from the top of the foot and a threaded portion in between the first end and the second end. An insert captured on the stem. The insert is threadably engaged to the stem by a generally cylindrical body with a threaded passage extending through the body adapted to matingly engage the threaded portion of the stem. A cap fixed on the second end of the stem to capture the insert. The insert disposed in a generally vertical passageway formed in the furniture and fixed therein to secure the glide assembly to the leg. The insert recessed into the leg to prevent prying or forcible removal without destruction of the furniture. While the tamper resistant guide securely hold down a piece of furniture, it does not have any hidden ligature functionality.

Norix Group Inc. makes a product line of ligature resistant beds. The beds are advertised as being used by behavioral healthcare professionals where traditional furniture is completely unsuitable for critical environments. The Norix bed is designed to provide the strength, safety and cleanability required for the daily rigors of institutional living, molded in specially formulated, high-impact, fire-retardant polyethylene, structurally enhanced for increased strength and durability. These beds feature seamless construction with rounded corners for added safety and security. The beds are designed to be attached flush to the wall or floor. However, the anti-ligature nature of the bed is more in the nature of having no ligature points at all on the furniture. Nothing in the Norix product line provides for a restraint point on the furniture or elsewhere such that it can be conveniently hidden and made inaccessible when not in use.

Thus, a need exists in the art for an improved restraint anchor that provides an improved restraint securing mechanism which is safe for use in a clinical environment and in which anti-ligature aspects are required when not in use.

### BRIEF SUMMARY OF THE INVENTION

Accordingly, in one particularly preferred embodiment, An anti-ligature device for use with restraints, is provided, the restraints used within an environment, the environment has a mounting location, the mounting location including a hole into which the anti-ligature device is mounted, the anti-ligature device including: a tub, the tub being open at one end, the tub has an outer surface and an inner surface, the tub inner surface partially defining an inner cavity, the tub being dimensioned for insertion into the mounting hole such that the tub fits within the hole, the tub outer surface being inaccessible from the environment when the tub is inserted into the hole; a tub securing element, the tub securing element secured to the inner surface of the tub and the mounting location; a compressible element has a first end and a second end, the compressible element disposed within the inner cavity of the tub and coupled at the first end to the tub; a tub sealing element, the tub sealing element has a restraint element for engaging the restraints, the tub sealing element coupled to the compressible element at the second end, the tub sealing element has an insertion portion and a top sealing portion, the top sealing portion of the tub sealing element coupled to the insertion portion, the insertion portion compressing the compressible element when the tub sealing element is inserted into the tub such that the tub sealing element is under force by action of the compressible element when inserted into the tub; at least a portion of the tub sealing element being exposed to the environment in an open position of the anti-ligature device, the restraint element for engaging the restraints being accessible in the open position, and the tub sealing element being inserted into the tub in a closed position of the anti-ligature device, the restraint element being disposed entirely with the tub in the closed position, and the tub sealing portion of the tub sealing element sealing the open end of the tub in the closed position.

In other particular preferred variations of the device, the device further provides the environment is a room and the restraint is a restraining strap for restraining a person, the restraint element is a slot for receiving the restraining strap, and the mounting location within the environment is one of a floor, a wall, or a piece of furniture; or the tub has a top surface at the open end, the top sealing portion of the tub sealing element has a top surface, the top surface of the tub and the top surface of the tub sealing element meeting to provide the seal between the tub sealing element sealing and the open end of the tub in the closed position; or the tub has a top surface at the open end, the open end of the tub has a circumference profile, the top sealing portion of the tub sealing element has a top surface, the top surface of the top sealing portion has a circumference profile, the circumference profiles of the open end of the tub and the tub sealing element being identical in shape, the circumference profile of the open end of the tub being slightly larger than the circumference profile of the tub sealing element, the top sealing portion of the tub sealing element fitting within the open end of the tub in the closed position such that the top surface of the tub is substantially coplanar with the top surface of the tub sealing element, the seal being provided by a continuous top surface of the anti-ligature device; or the tub is a cylindrical tub, the top sealing portion of the tub sealing element is a cylindrical lid, the insertion portion of the tub sealing element is an insertion cylinder, the insertion cylinder coupled to the cylindrical lid and has and insertion cylinder circumference, the lid circumference being greater than the insertion cylinder circumference such that a lip portion of the cylindrical lid is formed as defined by a portion of the cylindrical lid that extends outside the insertion cylinder circumference.

In yet other particular preferred variations of the device, the device further provides the tub sealing element is rotatable with respect to the tub, the insertion portion of the tub sealing element has at least one engagement pin attached to the insertion portion, the tub has at least one engagement slot, the engagement pin sliding within the engagement slot between a first position associated with the closed position of the anti-ligature device and a second position associated with the open position of the anti-ligature device; or the tub sealing element has at least one lock, the lock has an engagement element that secures the tub sealing element to the tub at a locking element within the tub when the anti-ligature device is in the closed position; or the at least one lock includes at least one locking pin coupled to the insertion portion, the tub includes at least one key hole on the inner surface of the tub, the tub sealing element includes a keyway coupled to the locking pin, the keyway disposed on the top surface of the tub sealing element, the keyway used to engage the at least one locking pin in the key hole in the tub when the tub sealing element is in the fully inserted position and the keyway is activated; or the at least one lock includes at least one locking pin coupled to the insertion portion and a keyway coupled to the locking pin, the keyway disposed on the top surface of the tub sealing element; or the compressible element is a spring, the bottom portion of the tub has a groove on the interior surface at a bottom portion of the tub, the first end of the spring being coupled to the bottom portion of the tub by being seated within the groove within the tub, the insertion portion of the tub sealing element has a lower surface, the lower surface has a groove, the spring coupled to the lower surface of the insertion portion by being seated within the groove on the lower surface of the insertion portion; or the tub securing element is a threaded rod, the bottom portion of the tub includes a threaded hole through which the threaded rod is coupled, the mounting location hole has is a double expansion-type anchor, the double expansion-type anchor is secured to the environment when the threaded rod is engaged with the double expansion-type anchor.

In a particularly preferred method according to the present invention a method of locking an anti-ligature device is provided, including the steps of pushing the tub sealing element at the top surface to release the tub sealing element from the first position; rotating the tub sealing element in relation to the tub; releasing the tub sealing element to allow the compressible element to force the tub sealing element and the device to attain the second position and expose the slot to the environment; securing a restraint using the slot; unsecuring the restraint from the slot; pushing the tub sealing element at the top surface; rotating the tub sealing element in relation to the tub to force a compression of the compressible element and cause the tub sealing element and the device to attain the first position; and locking the device in the first position by engaging the lock.

The objects and features of the present invention may be applied jointly or severally in any combination or sub-combination by those skilled in the art.

### BRIEF DESCRIPTION OF THE SEVERAL VIEWS OF THE DRAWING(S)

Detailed embodiments of the present invention(s) are disclosed herein; however, it is to be understood that the disclosed embodiments are merely exemplary of the invention(s), which may be embodied in various forms. Therefore, specific structural and functional details disclosed herein are not to be interpreted as limiting, but merely as a basis for the claims and as a representative basis for teaching one skilled in the art to variously employ the present invention(s) in virtually any appropriately detailed structure. Further, the terms and phrases used herein are not intended to be limiting, but rather to provide an understandable description of the invention(s).

The accompanying drawings, which are incorporated in and constitute part of this specification, illustrate embodiments and/or aspects of the invention and/or disclosure, and together with the written description, these drawings serve to explain the principles of the invention. Wherever possible, the same reference numbers are used throughout the drawings to refer to the same or like elements of an embodiment. The embodiments illustrated herein are presently preferred, it being understood, however, that the invention is not limited to the precise arrangements and instrumentalities shown, in which:

FIG. 1 shows various views of the apparatus according to one embodiment of the present invention;

FIG. 1A shows a top view of the apparatus according to one embodiment of the present invention;

FIG. 1B shows a front views of the apparatus in the closed position according to one embodiment of the present invention;

FIG. 1C shows a side view of the apparatus in the open position according to one embodiment of the present invention;

FIG. 1D shows a front views of the apparatus the open position according to one embodiment of the present invention;

FIG. 2 shows various views of a bed in an environment in which the present invention may be used;

FIG. 2A shows the top view of a bed in an environment in which the present invention may be used;

FIG. 2B shows the side view of a bed in an environment in which the present invention may be used;

FIG. 3 shows a full front view of the device according to one prototype embodiment of the invention;

FIG. 4A shows the side view of the tub according to one embodiment of the present invention;

FIG. 4B shows the oblique view of the tub according to one embodiment of the present invention;

FIG. 4C shows the top view of the tub according to one embodiment of the present invention;

FIG. 5 shows a top view of parts of the tub sealing element according to one embodiment of the present invention;

FIG. 6 various views of the locking pins according to one embodiment of the present invention;

FIG. 6A shows the bottom view of the locking pins according to one embodiment of the present invention;

FIG. 6B shows the side, view of the locking pins according to one embodiment of the present invention;

FIG. 6C shows the front view of the locking pins according to one embodiment of the present invention;

FIG. 6D shows the oblique views of the locking pins according to one embodiment of the present invention;

FIG. 7 shows various views of the securing cap according to one embodiment of the present invention;

FIG. 7A shows the top, view of the securing cap according to one embodiment of the present invention;

FIG. 7B shows the oblique view of the securing cap according to one embodiment of the present invention;

FIG. 7C shows the side view of the securing cap according to one embodiment of the present invention;

FIG. 8 shows various views of the keyway lock element according to one embodiment of the present invention;

FIG. 8A shows the side view of the keyway lock element according to one embodiment of the present invention;

FIG. 8B shows the top view of the keyway lock element according to one embodiment of the present invention;

FIG. 8C shows the bottom view of the keyway lock element according to one embodiment of the present invention;

FIG. 8C′ shows the oblique view the keyway lock element according to one embodiment of the present invention;

FIG. 8D shows an additional oblique view of the keyway lock element according to one embodiment of the present invention;

FIG. 8D′ shows an additional oblique view of the keyway lock element according to one embodiment of the present invention;

FIG. 9 shows various views of the lock cup according to one embodiment of the present invention;

FIG. 9A shows the top view of the lock cup according to one embodiment of the present invention;

FIG. 9B shows the oblique view of the lock cup according to one embodiment of the present invention;

FIG. 9C shows the sectional view of the lock cup according to one embodiment of the present invention;

FIG. 9D shows side view of the lock cup according to one embodiment of the present invention;

FIG. 10A illustrates a disassembly drawing of the tub sealing element according to one embodiment of the present invention;

FIG. 10B illustrates a partially assembled drawing of the tub sealing element according to one embodiment of the present invention;

FIG. 10C illustrates a fully assembled drawing of the tub sealing element including the keyway lock and securing cap according to one embodiment of the present invention;

FIG. 10D illustrates a more fully assembled drawing of the tub sealing element including the keyway lock and securing cap according to one embodiment of the present invention;

FIG. 11 shows various views of the interior restraint element according to one embodiment of the present invention;

FIG. 11A shows the top view of the interior restraint element according to one embodiment of the present invention;

FIG. 11B shows the bottom view of the interior restraint element according to one embodiment of the present invention;

FIG. 11C shows the sectional view of the interior restraint element according to one embodiment of the present invention;

FIG. 11D shows the side view of the interior restraint element according to one embodiment of the present invention;

FIG. 12 illustrates a partial assembly drawing of the tub sealing element in one stage of assembly according to one embodiment of the present invention; and

FIG. 13A illustrates the spanner key according to one embodiment of the present invention;

FIG. 13B illustrates the spanner key inserted into the assembly according to one embodiment of the present invention;

FIG. 14A shows an oblique view of the restraint anchor, in a closed configuration including the inserted spanner key, according to a first alternative embodiment of the invention;

FIG. 14B shows an oblique view of the restraint anchor, in an open configuration including the inserted spanner key, according to a first alternative embodiment of the invention;

FIG. 15 shows the top view of the restraint anchor according to a first alternative embodiment of the invention;

FIG. 16 shows the bottom view of the restraint anchor according to a first alternative embodiment of the invention;

FIG. 17 shows the front view of the restraint anchor according to a first alternative embodiment of the invention;

FIG. 18 shows an oblique view of the tub and compressible element according to a first alternative embodiment of the invention;

FIG. 19 shows a closeup of the tub bottom showing the inner volume according to a first alternative embodiment of the invention;

FIG. 20 shows an oblique view of the tub sealing element according to a first alternative embodiment of the invention;

FIG. 21 shows a side view of the tub sealing element according to a first alternative embodiment of the invention;

FIG. 22 shows a bottom view of the tub sealing element according to a first alternative embodiment of the invention;

FIG. 23 shows a oblique view of the restraint anchor in the open configuration and displaying the locking pins extended according to a first alternative embodiment of the invention;

FIG. 24 shows a side view of the restraint anchor in an open configuration according to a second alternative embodiment of the invention;

FIG. 25 shows an oblique view of the restraint anchor in a closed configuration according to a second alternative embodiment of the invention;

FIG. 26A shows the top view of the restraint anchor according to a second alternative embodiment of the invention;

FIG. 26B shows the bottom view of the restraint anchor according to a second alternative embodiment of the invention;

FIG. 27 shows a disassembly of the restraint anchor exposing the inner surfaces of the restraint anchor according to a second alternative embodiment of the invention;

FIG. 28 shows a disassembly of the restraint anchor exposing the exterior surfaces of the restraint anchor according to a second alternative embodiment of the invention;

FIG. 29 shows a closeup of the tub bottom showing the inner volume according to a second alternative embodiment of the invention;

FIG. 30 shows another closeup of the tub bottom showing the inner volume according to a second alternative embodiment of the invention;

FIG. 31 shows an oblique view of the tub sealing element according to a second alternative embodiment of the invention;

FIG. 32 shows a side view of the tub sealing element with spanner key inserted according to a second alternative embodiment of the invention;

FIG. 33 shows a side view of the restraint anchor in the open configuration with the spanner key inserted according to a second alternative embodiment of the invention;

FIG. 34 shows a closeup side view of the restraint anchor in the open configuration with the spanner key inserted according to a second alternative embodiment of the invention;

FIG. 35 shows an oblique view of the restraint anchor in a closed configuration with the spanner key inserted according to a second alternative embodiment of the invention;

FIG. 36A shows a side view of the restraint anchor in the open configuration with the spanner key inserted according to a third alternative embodiment of the invention; and

FIG. 36B shows a side view of the restraint anchor in the closed configuration respectively with the spanner key inserted according to a third alternative embodiment of the invention.

### DETAILED DESCRIPTION OF THE INVENTION

To facilitate a clear understanding of the present invention, illustrative examples are provided herein which describe certain aspects of the invention. However, it is to be appreciated that these illustrations are not meant to limit the scope of the invention and are provided herein to illustrate certain concepts associated with the invention.

The entire description below is provided in the particular context of a patient restraint system for use in a hospital setting, and in particular a strap restraint system used with mentally handicapped patients in a clinical and protective hospital room. However, the invention of the present application is generally applicable to any other restraint systems in which any object requires secure restraint in one situation and an “invisible locked and secure presence” in a second situation.

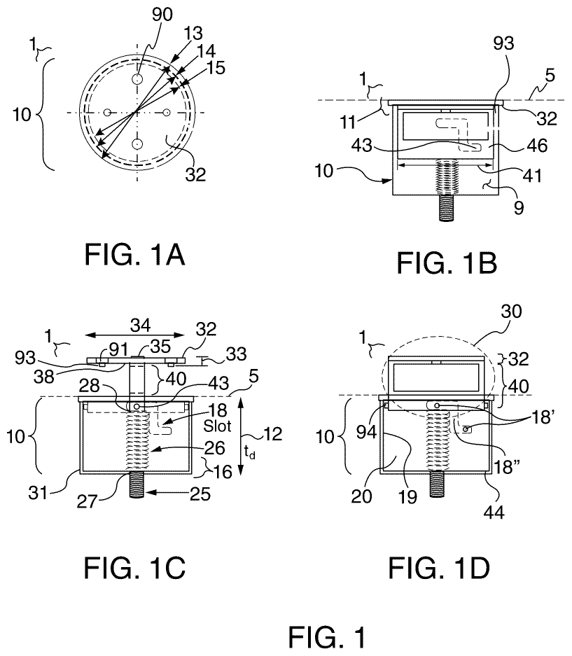

FIGS. 1A-1D show the anti-ligature device of the present invention in four different views (respectively): FIG. 1A a top view, FIG. 1B a front view (fully-inserted, closed or first position), FIG. 1C a side view (not fully-inserted, exposed or second position), and FIG. 1D another front view (not fully-inserted, exposed or second position). As shown, anti-ligature device 1 includes a tub 10, shown as a cylindrical enclosure. Tub 10 contains most of the other device components and is coupled to the other elements of the present invention. Tub 10 has outer surface 9, extending a depth to 12 and having an external tub diameter 14. Tub 10 includes a lip 11 at the top of the top. Lip 11 extends radially beyond tub outer surface 9 to a lip diameter 13 that is greater than the tub diameter 14. Tub 10 includes an inner volume 20 that is defined by the tub inner surface 19 having an inner tub diameter 15. Slot 18 is provided on tub 10 and extends through the entire tub, from inner to outer surface, so that a channel is formed within tub 10. The channel provides for access between the exterior of tub 10 and the inner volume 20. Preferably, slot 18 is geometrically configured in the shape of an “S,” a “Z” or a “C” as viewed from the tub side such that there are three portions of the slot—a first and a second (or top and bottom respectively) generally horizontal slot portions 18′ and one, generally vertical, slot portion 18″ conjoining the two horizontal slot portions.

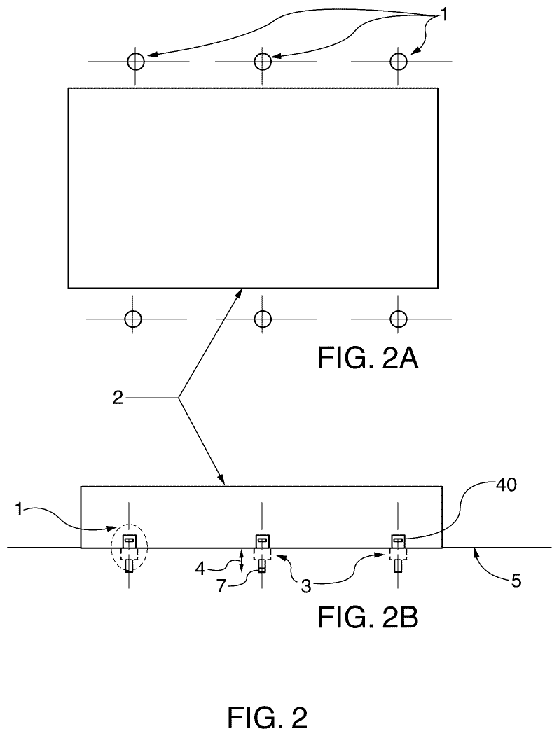

Tub 10 sits securely within a hole or mounting location 3. FIG. 2. Tub securing element 25 is coupled to a tub bottom portion 16 of tub 10. Tub securing element 25 is secured to the environment within the mounting location. In one preferred embodiment, tub securing element is a threaded screw or bolt that is secured to the bottom portion 16 of tub 10. In this arrangement, the bottom portion 16 of tub 10 may have a threaded nut encased as part of the tub structure for threadable engagement with the tub securing element 25. The environment (hole) may itself have a hole or a depression in which some type of anchor 7 may be disposed. A double expansion anchor maybe be embedded when the mounting hole is concrete or other hard substance. A straight bolt and washer may also be used on wood surfaces and other solid objects that have access to the back side of the object. When the threaded screw or bolt is placed in the hole and engaged with the double expansion type anchor, an outer portion of the anchor expands to fill the embedded location in which it sits and the threaded bolt then pulls the inner portion of the double expansion anchor into a secure adherence position such that tub 10 then sits securely anchored in mounting location 3.

Mounting location 3 can be a wall, a floor or other solid object of some depth and/or securing capability such that tub 10 and tub securing element 25 may be used to anchor the tub in a somewhat permanent fashion within that solid element. Flat mounting surface 5 is necessarily a part of the solid object and mounting location 3. The top surface of tub lip 11 is necessarily seated below the flat mounting surface 5, or at a minimum is flush with flat mounting surface 5 when the tub 10 is fully inserted into mounting location 3 and secured with tub securing element 25. This is important so that no portion of the tub 10 is accessible, bendable, pryable or otherwise capable of deformation or removal by the patient in the hospital room. In this regard, and in an ideal arrangement, the top surface of tub lip 11 is not only flush with flat mounting surface 5, but lip diameter 13 of lip 11 is dimensioned to precisely fit within any indentation made within the mounting location to accommodate tub 10 and lip 11 so that a continuous and smooth surface is achieved when the tub 10 is fully inserted into mounting location 3 and secured with tub securing element 25.

Compressible element 26 is disposed within inner volume 20 and coupled to bottom portion 16 of tub 10 at a first end 27. Compressible element 26 is coupled at a second end 28 to tub sealing element 30. Tub sealing element 30 is dimensioned to fit within inner volume 20 of tub 10. Compressible element 26 is compressed upon the insertion of tub sealing element 30 within inner volume 20 such that an upward force is applied to the tub sealing element 30 by the compressible element when the device is in the closed position and tub securing element 30 in its fully inserted position. In FIG. 1B, the device is shown in the first position, with the tub sealing element 30 in a fully inserted (closed position). In FIG. 1D, the device is shown in the second position, with the tub sealing element not fully-inserted (open position) and at least partially exposed to the environment.

Tub sealing element 30 is generally comprised of two portions coupled together: an insertion portion 40 and top sealing portion 32. Insertion portion 40 is coupled to the second end 28 of the compression element 26 at its bottom and to the top sealing portion 32 at a lower surface 33 thereof. As shown in FIGS. 1A-1D, insertion portion 40 also has at least one dimension that has an outer diameter 34 that is slightly smaller than inner tub diameter 15. In the embodiment illustrated in FIGS. 1A-1D, insertion portion 40 is cylindrical as viewed from the top (FIG. 1A) and has a generally rectangular solid form 42 as viewed from the front (FIGS. 1B, 1D). Insertion portion 40 has curved outer edge surfaces and a rectangular hole or slot 46 within the rectangular solid 42. In FIG. 1C, the side view of the rectangular solid comprising the insertion portion 40 is shown. In FIG. 1B, a front view of the rectangular insertion portion is shown as fully inserted into inner volume 20 of tub 10.

As shown in FIG. 1A, top sealing portion 32 is circular with a shallow depth 33 so as to comprise a shallow cylinder. Top sealing portion has an outer diameter that fits comfortably but snugly within inner lip diameter/outer tub diameter 14 when the tub sealing element is fully inserted into tub 10. Top sealing portion 32 includes locking pins 93 on the outer periphery of the cylinder. Locking pins 93 may be solid non-compressible protrusions that extrude from the outer periphery of the top sealing portion 32. One or more keyways 90 may be coupled to said locking pins may be included within or on the top of top sealing portion 32. Keyways 90 are rotatable such that rotation in one direction retracts the locking pins within the top sealing portion and rotation in the other direction extends them out beyond the periphery of the outer diameter 34 of the top sealing portion 32. Locking holes 94 are provided as indents on the inner tub surface 19 and are disposed at certain registered locations at the top portion of the tub 10. When the device is in the final, first closed position, the locking pins 93 are aligned with the locking holes 94 and the keyway may be turned to extend the locking pins into the locking holes and secure the entire tub securing element in a locked relationship with tub 10.

Engagement pins 43 are included as part of the structure of the insertion portion 40 and are slidably inserted into holes in the profile edges of insertion portion 40, preferably at a bottom portion thereof and slightly above the point at which the insertion portion is coupled to the second end 28 of compressible element 26. Springs may be used behind the engagement pins 43 so that the pins are naturally pushed out and away from the body of the insertion portion 40. When the top sealing portion 32 is fully inserted into tub 10, and in the fully assembled device arrangement, the engagement pins are aligned with the first horizontal slot portion upon insertion of the tub sealing element. As the tub insertion element is first inserted into tub 10, the pins 43 “pop out” when they engage with the top-most, first horizontal slot portion 18′. Once assembled, and both engagement pins 43 (one on each of the two sides of insertion portion 40) “pop out” and engage with slots 18, the anti-ligature device is fully assembled. Once fully assembled, pins 43 remain engaged in slots 18 for the duration of the operational life of the anti-ligature device, traversing the slots from one horizontal portion, through the vertical portion and then through the other horizontal portion. Engagement pins 43 may also be “T-shaped” as viewed from the side of the pin (pin profile). In this arrangement, they are of sufficient diameter to be positioned in the slot 18 and securably screwed into insertion portion 40 of the tub securing element 25 as permanently affixed within tub 10 so as to create the final assembly.

The anti-ligature is operable between the second position shown in FIG. 1D to the first position shown in FIG. 1B in the following manner. Transitioning the anti-ligature device is accomplished by pushing the tub sealing portion 32 down at the top surface 35, against the upward force exerted by the compression element 26. Simultaneous with the exertion of downward force, tub sealing element is rotated counter-clockwise (as viewed from the top) in relation to tub 10 so that engagement pins 43 travel along the upper horizontal slot portion 18′ over to the joinder location of slot portion 18 with vertical slot portion 18″. Continuing with the application of downward force, tub sealing element 30 is pushed into tub 10 and engagement pins 43 travel down along vertical slot portion 18″ of slot 18 and to the joinder of vertical slot portion 18″ with lower horizontal slot portion 18′. To complete the transition from the second device position to the first device position, the tub sealing element is rotated by the force placed on top surface 35 again simultaneous with the exertion of downward force, in a counter-clockwise (as viewed from the top) in relation to tub 10 so that engagement pins 43 travel along the lower horizontal slot portion 18′ over to the end of that horizontal slot portion.

It should be appreciated that in both the first (closed) and second (exposed) device positions the compression element applies an upward force on tub sealing element 30 at all times. When fully compressed, and the device is fully disposed in closed device position, the engagement pins 43 are forced against a top edge of the lower horizontal slot portion 18′. In this position, the compression element provides the greatest force on tub sealing element 30. When fully exposed, and the device is fully disposed in the second device position, the engagement pins 43 are forced against a top edge of the upper horizontal slot portion 18′. In this position, the compression element provides the least force on tub sealing element 30.

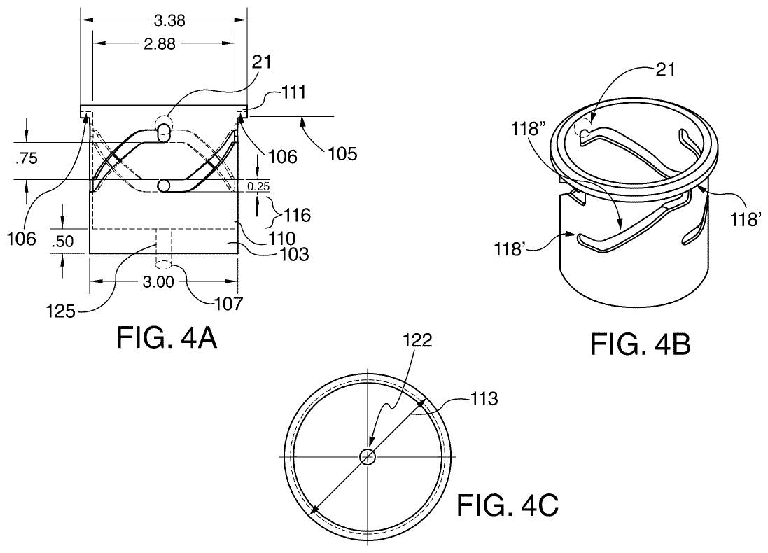

It should also be appreciated that the geometry of slot 18 can be any one of several shapes such that the above functions are accommodated. Thus an “S-shaped” or a “C-shaped slot” may be employed. In the case of a “C-shaped” slot the rotation direction of the tub sealing element would need to be reversed when transitioning between the two device positions. Most desirably, a “Z-shaped” slot is preferred so that the simultaneous application of force and rotation on the tub sealing element is more easily performed. Finally, registration notches 21 (FIG. 4A) may be included at terminal and interstitial points along the travel path of engagement pins 43 within slot 18 such that the transition may be partially completed and “stored” mid-way, or may otherwise be securely terminated at the ends of the slots. In fact, in experimental use, the registration notches 21 at the ends of slot 18 proved to be the most secure and safe method of securing the tub sealing element 30 in final resting positions and preventing inadvertent rotation of the tub sealing element when in those positions.

Is should be further be appreciated that different configurations and constructions of the tub and internal components may be achieved in accord with the teachings of the present invention. First, different tub geometries may be used. Square tubs, triangular tubs, truncated frustrum and other shapes of tubs are envisioned. Top portions of the top sealing portion may be maintained as round, and rotatably engageable within a round top hole in the top of an otherwise non-cylindrical tub. Different internal components and pin-hole or pin-slot arrangements may be used to accommodate variations to the basic geometric structures herein. The basic teachings provided herein are applicable, nonetheless. Further, the construction of the components may vary or be mixed and matched. The requisite parts may be cast metal, mild steel, with powder coating, stainless steel, high strength aluminum, molded plastic, 3D-printed material or combinations of any of the same, such that the objectives of the present invention are achieved. Also, different tub securing elements (hooks, clasps, hasps, rope, bolted platforms etc.) as well as different compression elements (pistons, hydraulics, deformable solids etc.) may be substituted so that the objectives of the invention herein are accomplished.

FIG. 2 shows an exemplary environment in which the anti-ligature device of the present invention is used. Patient bed 2 is provided disposed on a floor of the environment. The anti-ligature devices are embedded in the floor or mounting locations 3. In the hospital setting, the floor may be concrete and mounting holes 3 may be provided in the floor to a mounting depth 4. The flat mounting surface 5 is the floor surface. Tub sealing element 30 extends above the floor surface in the opened position so that the restraint element slot 46 is exposed to the environment. Straps may then be threaded through the one or more exposed restraint element slots and across the bed 2 so as to restrain the patient thereto.

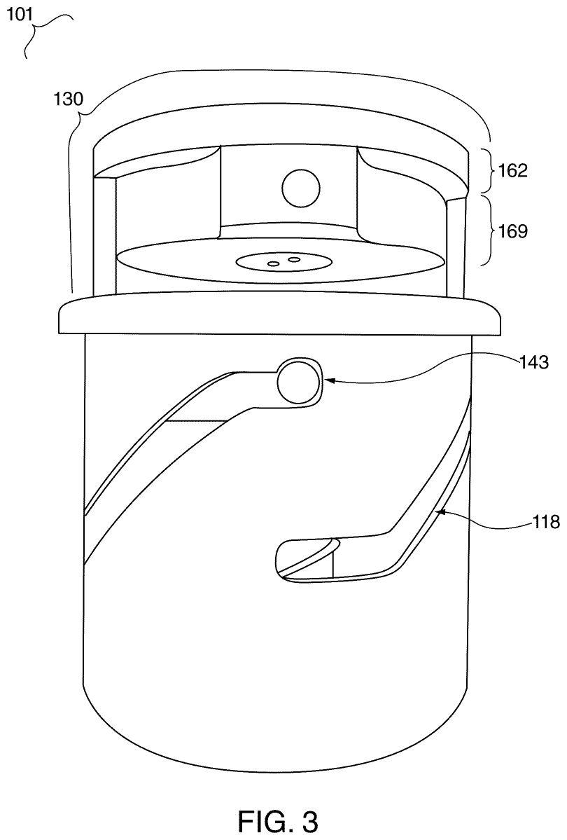

A particularly preferred prototype of the anti-ligature device of the present invention has been invented and is described herein in detail with respect to the following figures. FIG. 3 shows prototype anti-ligature device 101. In this embodiment, a mild steel construction having a powered coating is used for construction of the main structural pieces. When used in a floor and corrosion is of concern, a stainless steel construction may be desirable. In other applications high strength aluminum may be used as a fabrication material. FIG. 3 provides an oblique view of the device as fully assembled and displayed in the second (exposed, not fully-inserted) position.

FIGS. 4A-4C show, respectively, a front view, an oblique view, and a top view of the tub of the anti-ligature device according to one particularly preferred prototype embodiment of the present invention. Exemplary dimensions are included on the figures only as a reference as to the size of this embodiment (and are incorporated by reference to the FIGs. in the written portion of this specification in their entirety). FIG. 4A shows the tub 110 as it is inserted into mounting location (a hole in the floor) 103. The mounting location 103 is formed so that an annular recessed portion 106 of the mounting location is formed at mounting surface 105 and at the outside the diameter of the main mounting hole. Lip 111 of tub 110 then sits in that recessed annular region. In this position, the top of the top surface of the lip is flush with the mounting surface 105. Tub securing element 125 is shown as a threaded bolt that is secured to a bottom portion 116 of tub 110. Tub 110 has a threaded hole 122 in the center of the bottom thereof. As mentioned above, the mounting location 103 includes a recessed additional hole at the center bottom in which a double expansion anchor 107 maybe embedded. The dimensions of the tub 110 and the mounting location are tightly controlled and coordinated with respect to one another, and in particular the tub exterior diameter 114 and the diameter of the mounting location are matched so that a tight insertion fit is achieved when the tub 110 is fully inserted into the mounting location. In practical application, the tub may be inserted into mounting location without the tub sealing element 130 and the tub securing element 125 may be screwed “through” the threaded hole provided at the bottom of the bottom tub portion 116 of tub 110 and straight on through to a double expansion anchor 107 beneath the tub 110. Full insertion and tightening will then simultaneously expand and lock the double expansion anchor 107 to the mounting location 103 and secure tub 110 in place in that mounting location 103.

As shown in FIGS. 4A and 4B, four “zig-zag” or “Z-shaped” slots 118 are provided at the for ordinal positions of the tub, i.e. disposed evenly at 90 degrees from one another around the tub. Each slot 118 has two substantially horizontal portions 118′ and one substantially vertical portion 118″. As previously described, the engagement pins 143 attached to the tub sealing element engage in these slots to provide for slidable movement along the slots when the tub sealing element is transitioned from the first to the second positions and back again.

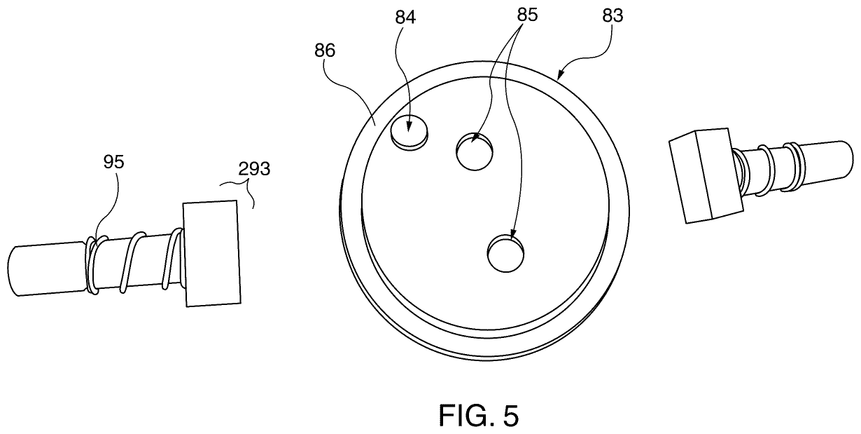

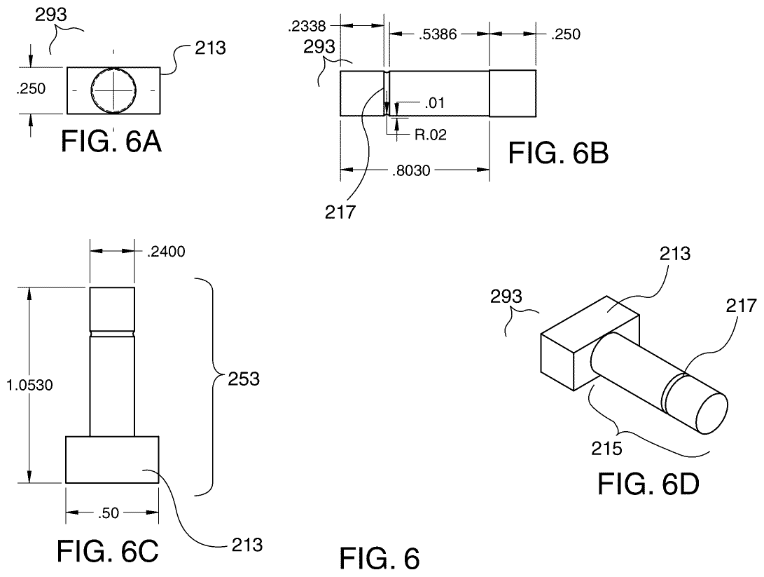

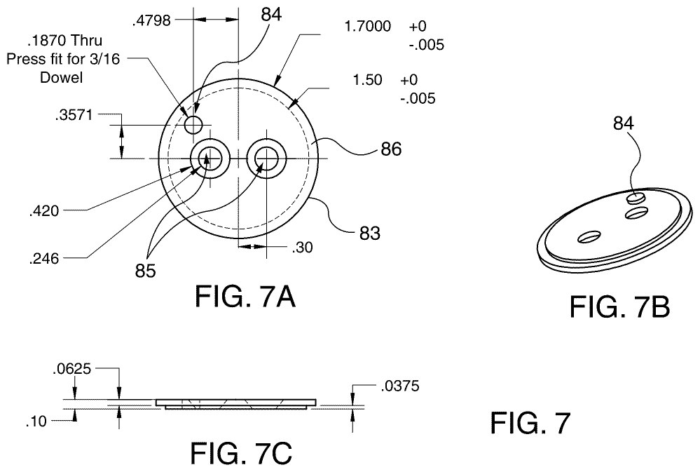

FIG. 5 shows locking pins 293, with springs 95 attached thereto and securing cap 83. Locking pins 293 are shown in greater detail in FIGS. 6A-6C. Securing cap 83 is shown in greater detail in FIGS. 7A-7C.

FIGS. 6A-6D show, respectively, a bottom view, a side view, a front view and an oblique view of the locking pins 293 according to one particularly preferred prototype embodiment of the present invention. Exemplary dimensions are included on the figures only as a reference as to the size of this embodiment (and are incorporated by reference to the FIGs. in the written portion of this specification in their entirety). Locking pins 293 include head end pin portion 213, pin stem 215 and ridge 217 on pin stem 215.

FIGS. 7A-7C show, respectively, a bottom view, an oblique view and a side view the securing cap according to one particularly preferred prototype embodiment of the present invention. Exemplary dimensions are included on the figures only as a reference as to the size of this embodiment (and are incorporated by reference to the FIGs. in the written portion of this specification in their entirety). Securing cap 83 includes recessed annular region 86, dowel turn stop 84, and through holes 85.

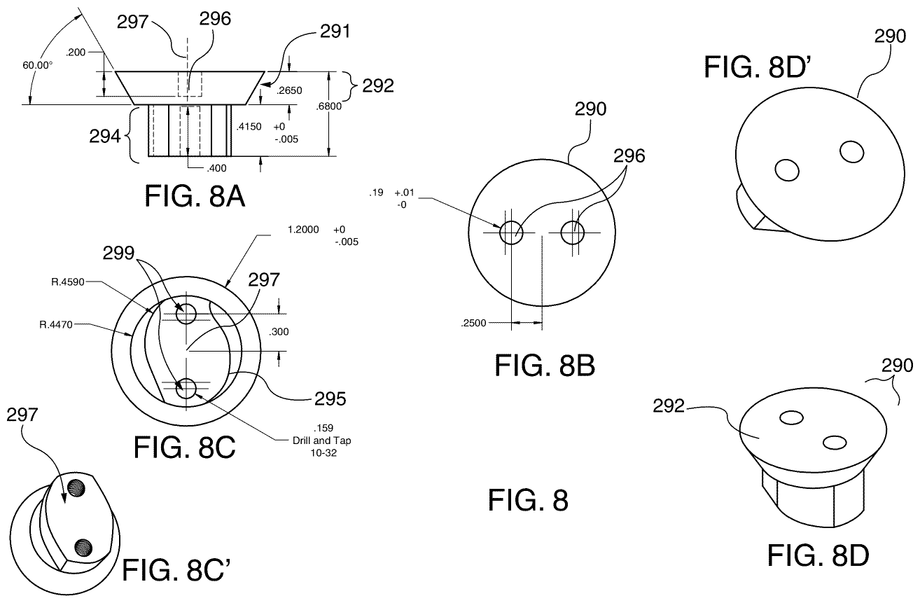

FIGS. 8A-8D show, respectively, a side view, a top view a bottom view and three different oblique views of the keyway lock element 290 according to one particularly preferred prototype embodiment of the present invention. Exemplary dimensions are included on the figures only as a reference as to the size of this embodiment (and are incorporated by reference to the FIGs. in the written portion of this specification in their entirety). Keyway lock element 290 includes a head portion 292 having a flanged or chamfered edge 291. The head portion 292 is coupled to key stem 294 and includes key holes 296. The key stem 294 has an undulating edge profile 295 such that its edge swoops in an out with respect to a central axis 297 of said keyway lock element 290.

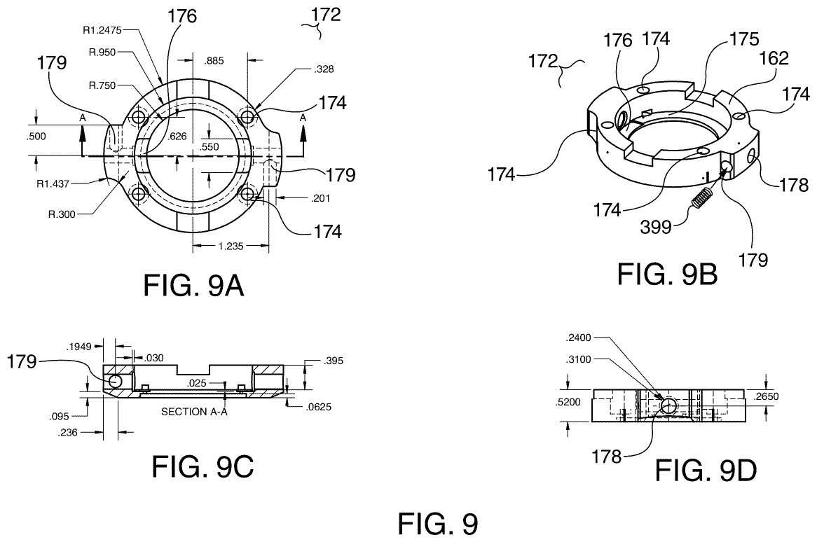

FIGS. 9A-9D show, respectively, a top view, an oblique view, a side sectional view and a side view of the lock cup 172 according to one particularly preferred prototype embodiment of the present invention. Exemplary dimensions are included on the figures only as a reference as to the size of this embodiment (and are incorporated by reference to the FIGs. in the written portion of this specification in their entirety). Lock cup 172 is generally annular in shape. The annular lock cup 172 includes mounting holes 174 drilled vertically therethrough. Lock cup 172 has an inner annular ledge 175 disposed at the bottom of the annular lock cup 172. The inner annular ledge 175 includes a locking pin seat 176. Locking pin holes 178 are drilled laterally through the side of lock cup 172 from the locking pin seat 176 to the lateral exterior wall of the lock cup 172. Securing pin holes 179 are included transverse to the locking pin holes 178 within the annular ring. Securing pin holes are 179 are preferably threaded and are drilled through to meet the locking pin holes 178 at a midpoint thereto. A threaded securing pin 399 is provided to be threadedly engaged in securing pin holes 179 so as to be able to contact locking pin holes 178. The securing pin is inserted into securing pin hole 179 so as to prevent locking pins 293 from retracting too far back into lock cap 172 by “catching” the pins at ridge 217.

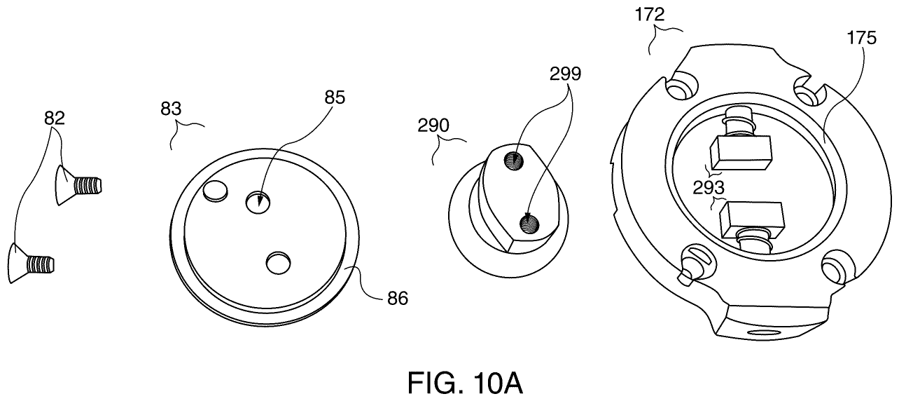

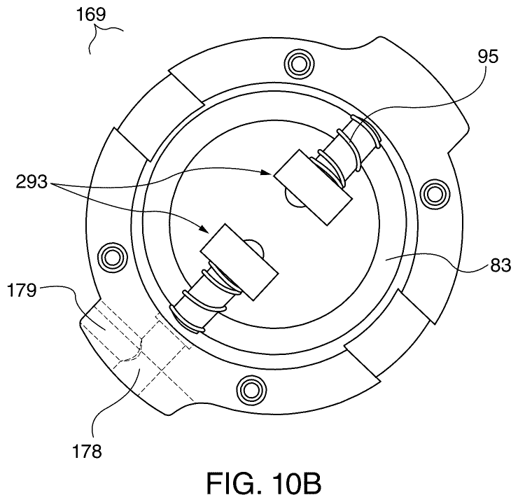

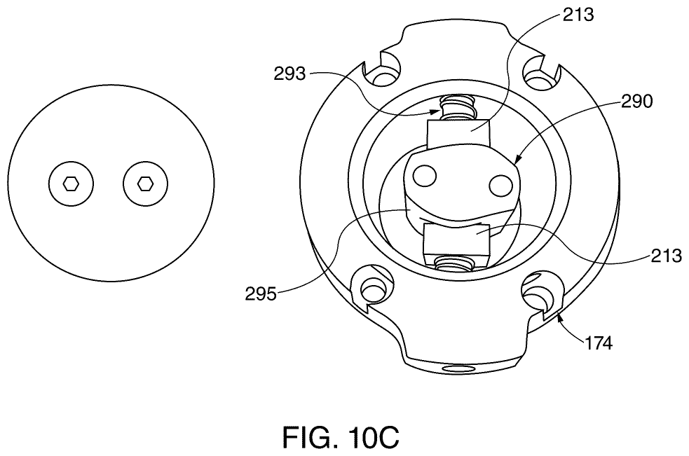

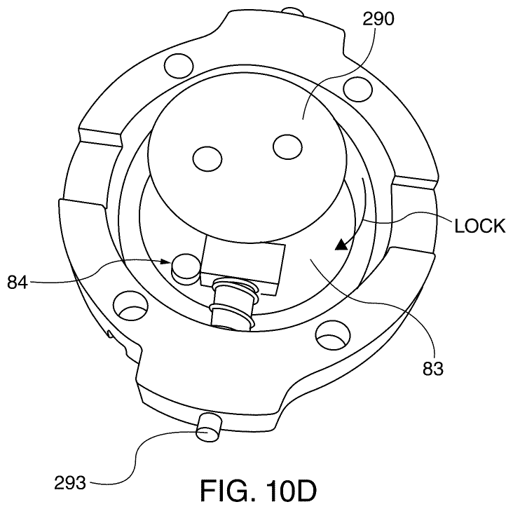

FIGS. 10A-10D illustrate a series of assembly drawings of the parts of the tub sealing element 130 in various stages of assembly according to one particularly preferred prototype embodiment of the present invention. In FIG. 10A, a bottom view of each of the unassembled assembly components is shown. Mounting screws 82 are positioned for insertion through mounting holes 85 in securing cap 83. Securing cap 83 is positioned for covering the bottom of lock cup 172 such that recessed annular region 86 rests against the downward facing inner annular ledge 175 of locking cap 172. The diameter of securing cup 83 matches the diameter of the inner edge of central annular area of locking cup 172. Keyway lock element 290 has keyway mounting holes 299 on the bottom of key stem 294 thereof for mateable coupling with mounting screws 82 when keyway lock element—turned upside-upside down—is inserted into the top of the annular ring (i.e. from the backside of lock cap 172 as shown in FIG. 10A).

In FIG. 10B, a top view is shown of the upper restraint assembly 169 is shown including the following components: locking pins 293 and securing cap 83. Locking pins 293 have the springs 95 inserted over the pin stem 215 and are inserted into the locking pin holes 178.

In FIG. 10C, a bottom view is shown of the partially assembled tub sealing element 130 including the following components: locking pins 293 and keyway lock element 290. Locking pins 293 have the springs 95 inserted over the pin stem 215 and are inserted into the locking pin holes 178. Locking pin head ends 213 rest on the undulating edges of 295 of fully inserted keyway locking element 290. It should be appreciated that the assembly of FIG. 10C omits the interposing interior restraint element onto the bottom of which is affixed the lock cup 172 and the top of which accommodates the keyway lock element 290. It should also be noted that mounting holes 174 are accessed for assembly from the underside of the tub sealing element and through access holes in the bottom of the interior restraint element.

In FIG. 10D, a top view is shown of the partially assembled tub sealing element 130 as provided in FIG. 10C but further including the securing cup 83 assembled thereto at the bottom surface. Again, the following components are included: locking pins 293 and keyway lock element 290. Locking pins 293 have the springs 95 inserted over the pin stem 215 and are inserted into the locking pin holes 178. Locking pin head ends rest on the undulating edges of 295 of fully inserted keyway lock element 290. It should be appreciated that in one rotational position of the keyway lock element the undulating edges of key stem 294 exert force on the pin heads end 213 causing the locking pins 293 to be pushed through the locking holes 294 and out the sides of the annular region of the lock cup 172. In this position, the springs 95 exert force on the key stem 213 and cause push back force into the annular ring. Dowel turn stop 84 provides for a stopping mechanism in the rotation of keyway lock element 290 so that the appropriate rotation of the keyway is provided to effect sufficient pin stem insertion without allowing the keyway lock element 290 from progressing past a maximal point of pin stem extension. Again, it should be appreciated that the assembly of FIG. 10D omits the interposing interior restraint element onto the bottom of which is affixed the lock cup 172 and the top of which accommodates the keyway lock element 290. This has been omitted to show clearly the workings of the keyway lock element rotation and engagement of the locking pins.

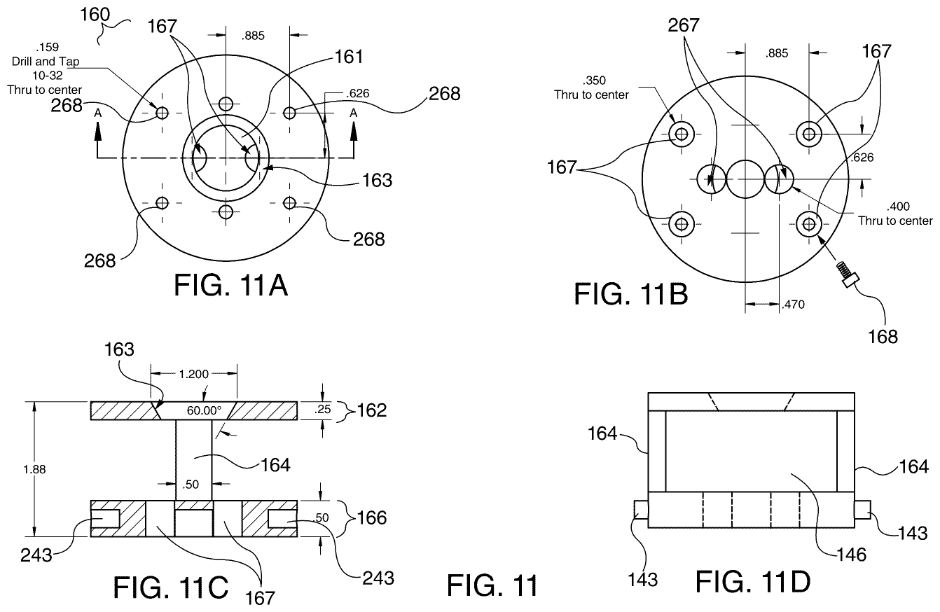

FIGS. 11A-11D show, respectively, a top view, a bottom view, and a section view and a front view the interior restraint element 160 according to one particularly preferred prototype embodiment of the present invention. Exemplary dimensions are included on the figures only as a reference as to the size of this embodiment (and are incorporated by reference to the FIGs. in the written portion of this specification in their entirety). Interior restraint element 160 is generally cylindrical as shown in the top view of FIG. 11A. Top restraint disk 162 has a hole in the middle of the top 161 with a chamfered edge 163 on the hole edge. Restraint sidewalls 164 are coupled to the top restraint disk at opposite sides of the top restraint disk 162. Only one restraint side wall is shown in the side view of FIG. 11C with the second being disposed directly behind the one being showing in that FIG. Bottom restraint disk 166 is coaxial with top restraint disk 162 and is of the same diameter and size. Along with the upper restraint assembly 169 (described below) restraint sidewalls 164 are coupled to the bottom restraint disk at opposite sides of the bottom restraint disk. Restraint disks 162 and 166, coupled by restraint sidewalls provide for an open form cylindrical element (in combination with all other attached elements—one tub sealing element 130) for insertion into tub 110.

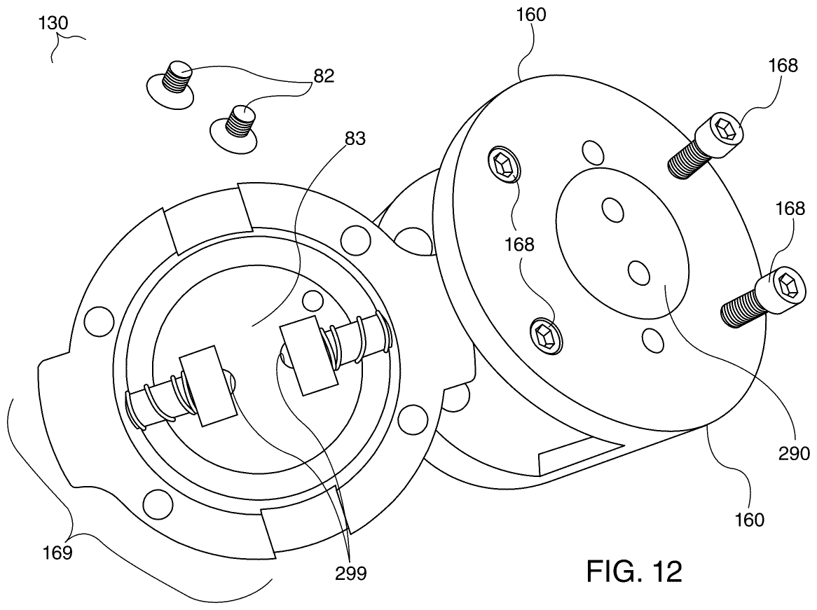

FIG. 11D shows a full front profile of the interior restraint element 160 including the restraint element slot 146. In a fully assembled tub sealing element, the lock cup 172, securing cap 183 and locking pins 93 (together upper restraint assembly 169) are all presented into the middle of interior restraint element 160 by insertion through slot 146 as shown in FIG. 12. After insertion of the lock cup and associated parts, the components are assembled as follows.

Four through holes 167 are provided on the bottom of bottom restraint disk 166. These through holes are dimensionally arranged the same as the four mounting holes 174 on the bottom of lock cup 172. The through holes 167 are provided to allow screwdriver/allen wrench access through the bottom restraint disk 166 and the restraint element slot 146 to the long mounting screws 168 shown in FIG. 12. Using long mounting screws 168, the upper restraint assembly 169 may be inserted into place and secured to the underside of the top restraint disk 162 of interior restraint element 160. Is should be appreciated that for the locking pins 93 to work, the upper restraint assembly ideally orients the locking pins pointing out the side of the interior restraint perpendicular to the restraint element slot 146, i.e. pointing straight out the sides as shown in FIG. 11D. Finally, once the long mounting screws 168 have been fully inserted there is a small space in the screw holes 268 in the top restraint disk 162. These small spaces may be sealed over with putty, glue or other composite material to prevent top-side access to the screw ends at the top surface of the top restraint disk, which sits exposed at the flush surface of the flat mounting surface 5.

Two through holes 267 are also provided on the bottom of bottom restraint disk 166. These through holes are arranged so as to register with the two mounting holes 85 on securing cap 83. The through holes 267 are provided to allow screwdriver/allen wrench access through the bottom restraint disk 166 and the restraint element slot 146 to the mounting screws 82 shown in FIG. 10A. Using mounting screws 82, the securing cap 83 may be inserted into place within the upper restraint assembly 169, simultaneously with the insertion of the keyway locking element 290 into chamfered hole 161 and secured to the keyway locking element 290 at keyway mounting holes 297. This assembly difficulty should be appreciated in that keyway locking element 290 is inserted into the top restraint disk 162, while simultaneously inserting the upper restraint assembly 169 into the interior restraint element 160 and aligning the undulating edges 295 of key stem 294 such that proper registration of all parts is made upon assembly.

Also shown in the sectional view FIG. 11C are two half sections of engagement pin receptacles 243. Engagement pin receptacles are configured for receiving engagement pins 143 as shown in FIG. 3. Again, these engagement pins are permanently attached to the tub sealing element 130, and once the tub sealing element 130 is inserted into the tub 110 they are permanently disposed within slots 118 on tub 110 for slidable operation of the device as it transitions between the exposed and closed positions.

FIG. 12 shows a partial assembly arrangement of the sealing element 130 the interior restraint element 160 having keyway locking element 290 inserted into chamfered hole 161 at the top surface thereof. Upper restraint assembly 169 is partially inserted into the restraint element slot 146 and long mounting screws 168 are positioned for securing the upper restraint assembly 169 to the interior restraint element 160 at the underside of top restraint disk 162. Next, mounting screws 82 are positioned for securing the securing cap 83 to the keyway locking element 290 at keyway mounting holes 299 from the bottom as viewed in FIG. 12. All mountings take place through access holes in the bottom restraint disk as previously described.

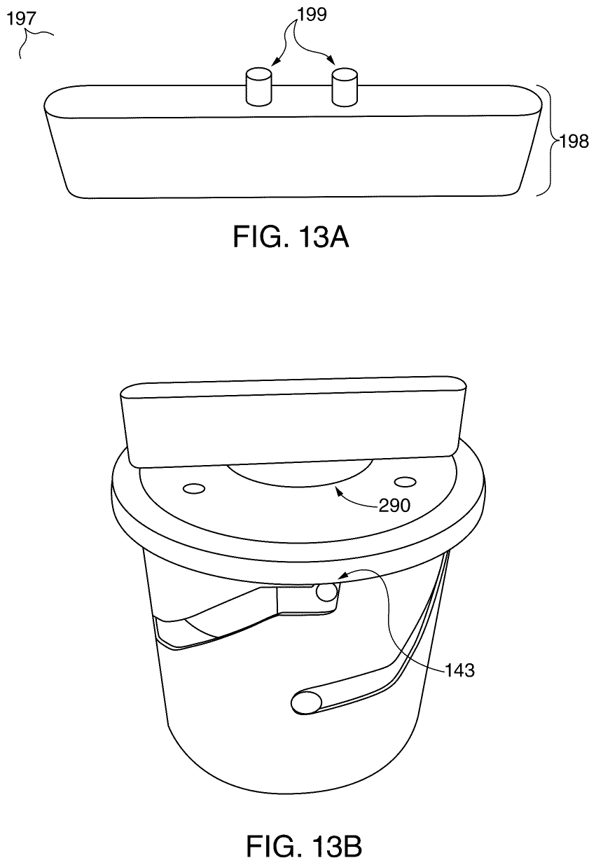

FIG. 13A show the spanner head key 197 used to rotate the keyway lock element 290 which throws the locking pins 293, via the operation of the undulating edge on 295 on key stem 294 so as to lock and unlock the tub sealing element 130 to the tub 110. In one particularly preferred embodiment, the locking pins 293 extrude out to the same tub holes/slot 118/registration notches 21 that are used for the engagement pins 143. Since locking only take place in the closed device position, the upper slot areas, particularly along the upper horizontal slot 118′ are left vacant for use of the same by the locking pins 293. Spanner head key 197 includes handle portion 198 and key pins 199. Key pins 199 are inserted into key holes 293 as shown in FIG. 13B showing the fully-assembled anti-ligature device 101 in the first position. Key holes 293 are shown at the top of keyway locking element 290 and rotated to engage the locking pins. In operation, the compressible element 126 provides sufficient force on the tub sealing element 130 such that the keyway lock element cannot easily be rotated without the spanner head key 197.

Following the initial development of the above-described restraint anchor device, numerous improvements have been made to address certain use and design considerations. The use considerations involved operational personnel having difficulties physically closing the device and identifying a “closure point” when completing the closure operation. In the design shown in FIGS. 1A-1D and FIG. 3, significant, upward resistance is provided by compressible element 26 when attempting to close the device. From a use standpoint, that resistance made it difficult for the operator to simultaneously push the tub sealing element 130 downward while using the spanner head key 198 to push and turn the entire mechanism to ultimately lock the restraint anchor device in the closed configuration. The most difficult action proved to be the final turning of the tub sealing element to lock the device, i.e. where the engagement pins traverse the final horizontal segments of the tub slots, that motion taking place under of the full pressure of the spring (the compressible element in the commercial embodiment). This places great pressure on the engagement pins against the tops of the lower horizontal slots making the full closure motion awkward and difficult. To compound matters, these difficulties were often accentuated by the need to simultaneously pay attention to a patient during the closure process.

From a design standpoint, the complexity of the locking functions housed within tub sealing element 130 made fabrication, maintenance and troubleshooting difficult to manage. Lock cap 172 has to be in strict alignment with tub sealing element 130. All the moving parts and springs, have to be manufactured and assembled according to strict tolerances, tested, potentially lubricated and are prone to wear over time. These parts include but are not limited to locking pins 293, keyway lock element 290, securing cap 83, springs 95, threaded securing pins 399 used to catch the locking pins 293. The initial overall design of the locking mechanism was complicated and required improvement.

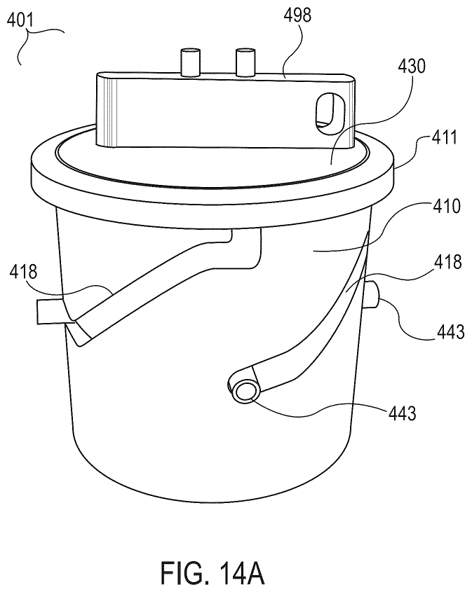

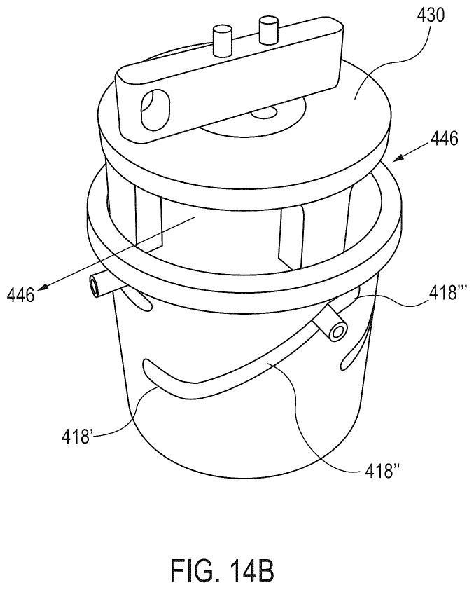

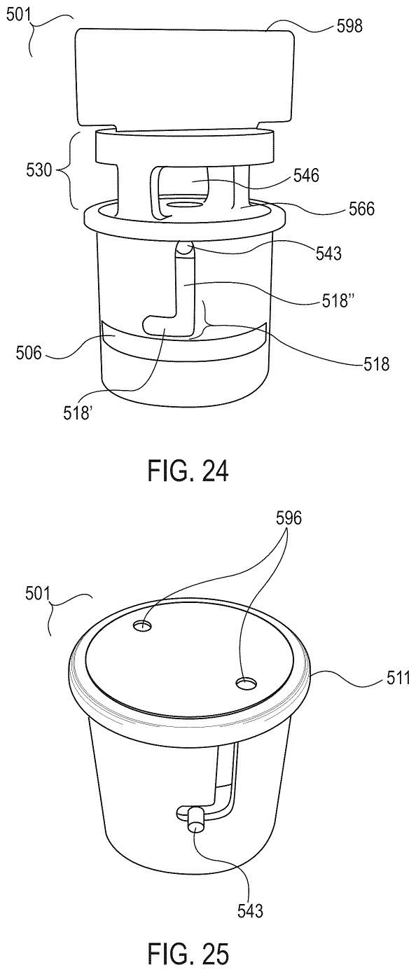

These real-world aspects resulted in design improvements that produced several alternative embodiments. FIGS. 14-23 provide a graphic representation of the restraint anchor device according to a first alternative embodiment of the invention. FIGS. 24-35 provide a graphic representation of the restraint anchor device according to a second alternative embodiment of the invention. FIGS. 36A and 36B provide a graphic representation of the restraint anchor device according to a third alternative embodiment of the invention.

FIG. 14A shows an oblique views of the restraint anchor device 401, in a closed configuration. FIG. 14B shows an oblique view of the restraint anchor device 401 in an open configuration. Tub 410 is provided in mild steel construction and includes lip 411 and slots 418. Bottom of lip 411 is designed to seat against an insertion barrier when tub 410 is inserted into a mounting location within the environment. In the closed configuration, tub sealing element 430 is shown recessed fully within the tub 410 such that the top surface of the tub sealing element 430 is flush with the top surface of lip 411. Spanner key 498 is shown as inserted into the key holes 496 in the top of tub sealing element 430 for use in rotating the tub sealing element 430 into the closed configuration. Slot 446 is provided as a through hole within the tub sealing element for receiving patient restraints.

In going from the closed configuration to the open configuration, spanner key 498 can be gripped by the restraint anchor user, rotated counterclockwise (from a top perspective), to rotate tub sealing element 430. Through the action of the engagement pins 443 moving through slots 418 the tub sealing element is first rotated counterclockwise until the engagement pins 443 reach, sequentially, the end of the bottom horizontal slot portion 418′, then through vertical slot portion 418″, and then through top horizontal slot portion 418′″. The action of the engagement pins 443 guided through the entire slot 418 causes the tub sealing element rise out of the tub 410 under the action of compressible element 426.

In other embodiments, the slots 418 may be eliminated and the tub sealing element 430 may simply be pushed down into tub 410. Any type of engagement mechanism can be attached to the bottom or sides of tub sealing element 430 that, when coupled with a matching engagement element within tub 410, cause the tub sealing element 430 to be clasped in the closed position with respect to tub 410 such that patient restraint receiver 446 is contained entirely within tub 410. Compressible element 426 may also be eliminated such that the spanner key 498 has its own coupling mechanism that couples to the tub sealing element 430 for gripping an moving the same under the action of the restraint user.

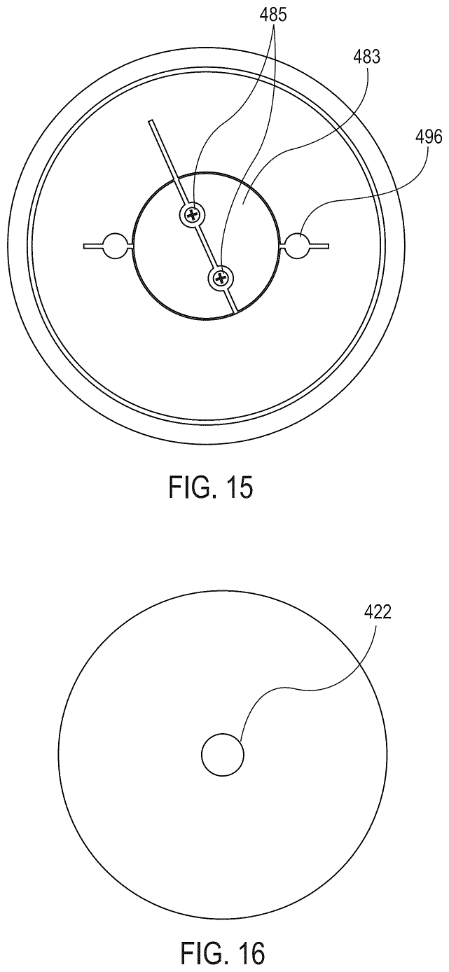

FIG. 15 shows the top view of the restraint anchor according to a first alternative embodiment of the invention. Securing cap 483 is shown within the top of tub sealing element 430. The upper surfaces of the tub sealing element 430, the securing cap 483 and the top surface of lip 411 provide one continuous and smooth surface to provide the anti-ligature nature of the restraint anchor 410 when in the closed configuration. Holes 485 are provided in securing cap 483 for rotating the securing cap 483 and activating lock cap 472 to extend the locking pins 493. Spanner key 498 may be configured and fabricated for dual engagement and rotation purposes by fabricating the pins on one side 499 to engage holes 485 in the securing cap 483 to extend locking pins 493 and on the other side 497 to engage the key holes 496 in the top surface of the tub sealing element 430 to securely depress the tub sealing element 430 into tub 410.

FIG. 16 shows the bottom view of the restraint anchor 401 according to a first alternative embodiment of the invention, including mounting hole 422, which may be threaded. Mounting hole 422 is used to securely mount the anti ligature device within the environment as previously described.

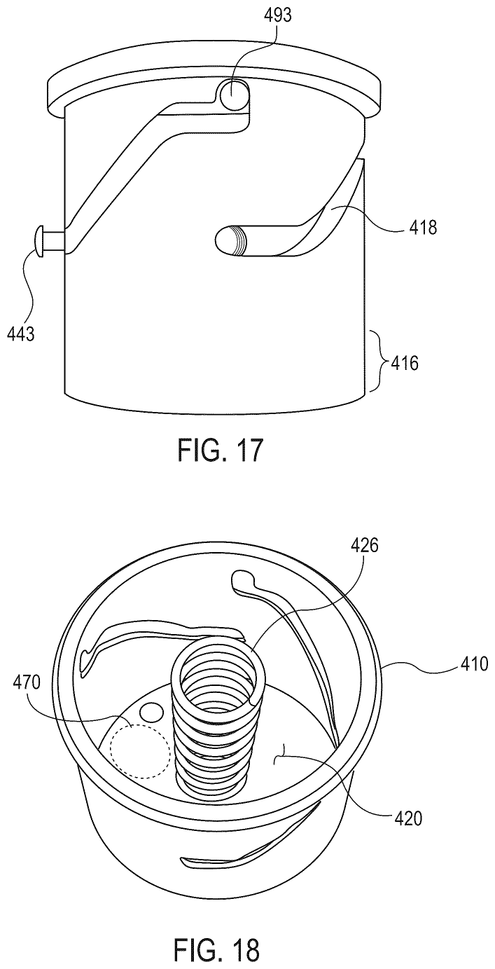

FIG. 17 shows the front view of the restraint anchor according to a first alternative embodiment of the invention. Here, one engagement pin is shown 443 while the engagement pin corresponding to the front-facing slot 418 has been removed (unscrewed or uninserted) from the tub sealing element 430 to reveal the hole (optionally threaded) within the tub sealing element 430 into which it is secured. Locking pin 493 is shown as not locked (not extended) in this closed configuration.

FIG. 18 shows an oblique view of the tub 410 and compressible element 426 according to a first alternative embodiment of the invention. Compressible element 426 is shown here as a helical spring positioned in the center of the inner volume 420 of tub 410.

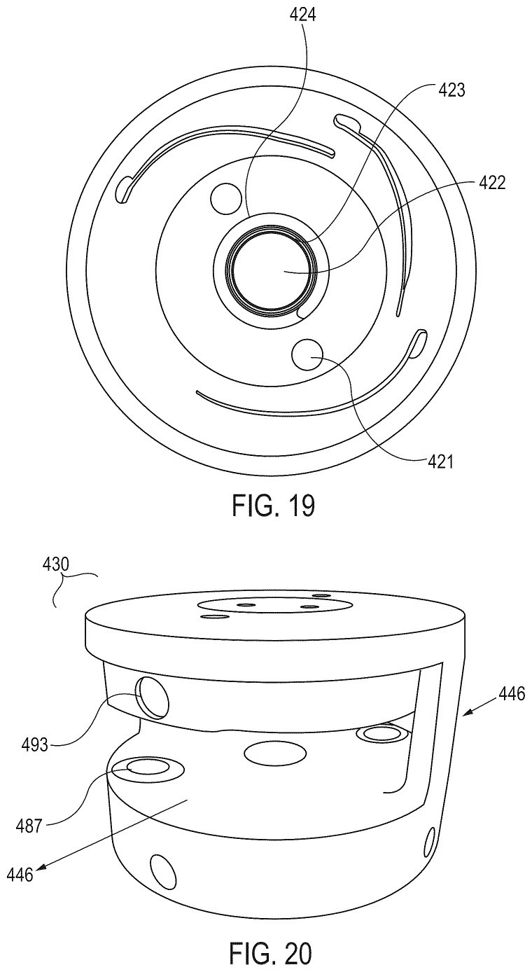

FIG. 19 shows a closeup of the tub bottom portion 416 within the inner volume 420 according to a first alternative embodiment of the invention. Mounting hole 422 is shown with threads 423. Annular ring 424 is shown and is milled out of the inner surface 419 of tub bottom portion 416 to provide a seat for compressible element 426. Divots or indents 421 are also milled out of inner surface 419 of tub bottom portion 416 and provide a seat for the ball nose 489 of plunger 487 in the final closed configuration of the restraint anchor as described below.

FIG. 20 shows an oblique view of the tub sealing element 430 according to a first alternative embodiment of the invention. Retracted locking pins 493 and slot 446, for insertion of the patient restraint, are clearly shown.

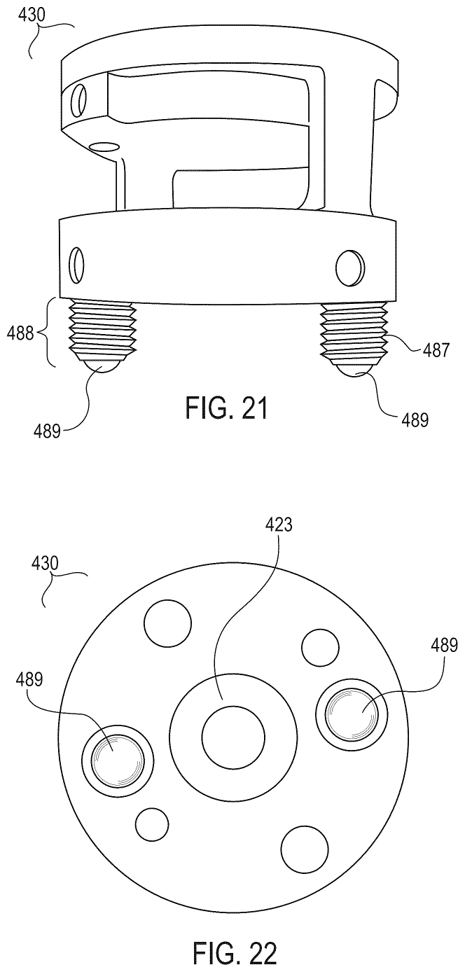

FIG. 21 shows a side view of the tub sealing element 430 displaying additional elements according to a first alternative embodiment of the invention. Specifically, plunger 487 is shown as being threadably engaged with the tub securing element 430. FIG. 20 shows the inserted side of plunger as screwed into the tub sealing element 430 with the bottom plane 487′ of the threaded end of plunger 487 being coplanar with the bottom surface of the restraint engagement slot 446. Referring to FIG. 21, plunger 487 extends below the bottom of tub sealing element 430 a certain distance 488. At the end of the plunger 487 is ball nose 489 that compresses into the plunger 487 when under force. Ball nose 489 is a metal ball that is situated within the plunger housing and is compressibly pushed within the plunger to extend beyond the plunger tip when no pressure is applied to the ball nose 489. Ball nose 489 is pushed into the plunger when a force is applied to the ball nose 489. The entirety of plunger 487 and 489 can be composed of any compressible element or combination of parts that result in a functionally compressible element.

One such plunger that may be used for this application is the McMaster-Carr high torque ball-nose plunger. These plungers are made with differing thread count for insertion and different nose force application pressures to accommodate different force insertion design criteria.

FIG. 22 shows a bottom view of the tub sealing element 430 according to a first alternative embodiment of the invention. Ball noses 489 are clearly shown there as extending out from the plungers 487. Further, upper recessed annular region 423 is shown milled and recessed within the bottom surface of tub sealing element 430. Annular reg 423 is sized and configured to accept and seat the top portion of compressible element 426.

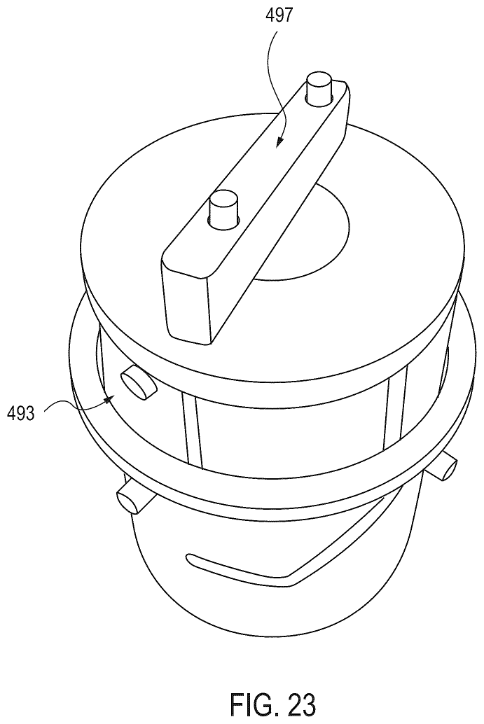

FIG. 23 shows a oblique view of the restraint anchor device 401 in the open configuration and displaying the locking pins 493 extended out the according to a first alternative embodiment of the invention. As shown in FIG. 17, the plurality of locking pins 493 are spaced around the perimeter at the top of tub sealing element 430 with such spacing that upon extension, they occupy the recess at the end of upper slot portion 418.′″

In operation the patient restraint device works as follows. In the open configuration, the ball noses 489 are in the extended position, with no pressure being exerted on the ball nose. As the tub sealing element 430 is pushed into the tub 410, the engagement pins 443 guide the tub sealing element 430 down and into the tub 410 and under the pressure of the compressible element 426. At the lowest point of travel though vertical slot portion 418″, just before the final horizontal travel segment along horizontal slot portion 418′ takes place, plungers 487 are close to the top surface of tub bottom 416 and the ball noses 489 are compressed a small distance within the plungers 487. As the ball noses 489 touch the top surface of the tub bottom, and under compression move along that top surface, the plungers 487 cause an additional upward pressure on the tub sealing element 430. The tub sealing element 430 is finally moved through the last horizontal slot segment 418′ where it stops at the end of the slot. This ball nose compression persists throughout horizontal travel through slot 418′ and to the end of the horizontal travel run. At that position, the divots 421 are placed in such a manner that they are aligned with the plunger 487 and the ball noses 489 extend back out of the plunger 487 to engage the divots at the end of the insertion operation. As appreciated by those of skill in the art, the sizing of the plunger 487 to the tub 410 dimensions, combine with the rotational location of the plunger 487 and divots 421 within the tub 410 and on the tub sealing element 430 are of critical importance so that the plunger bottoms are smooth with the upper surface of the slot 446 and the ball noses 489 are compressed by only a certain amount at the point of maximum insertion of tub sealing element 430 into tub 410.

This inner locking function using the plunger and divots allows for two advantageous functions. First, the tub sealing element is not loose or free to slide circularly when in the closed configuration. This is what is meant by locking in the sense that free movement of the tub sealing element is restrained. Second with the ball nose providing some upward pressure on the tub sealing element at maximal insertion the compression force of the compressible element may be reduced somewhat while the same upward force is maintained on the tub sealing element in the closed configuration. This in turn makes the closing function easier for the restraint anchor operator, i.e. less force is required to close the restraint anchor device through a majority of its operation. As mentioned, these additional safety features and ease-of-use enhancements are highly desirable in the complicated environment in which the restraint anchor operates.

FIGS. 24-35 provide a graphic representation of the restraint anchor device 501 according to a second alternative embodiment of the invention. FIG. 24 shows a side view of the restraint anchor device, in an open configuration. FIG. 25 shows an oblique view of the restraint anchor device 501 in a closed configuration. Tub 510 is provided in stainless steel construction and includes lip 511 and slot(s) 518. Bottom of lip 511 is designed to seat against an insertion barrier when tub 510 is inserted into a mounting location within the environment. In this configuration, tub sealing element 530 is shown recessed fully within the tub 510 such that the top surface of the tub sealing element 530 is flush with the top surface of lip 511. Spanner key 598 is shown as inserted into the key holes 596 in the top of tub sealing element 530 for use in rotating the tub sealing element 530 into the closed configuration. Slot 546 is provide as a through hole within the tub sealing element for receiving patient restraints. View slot 506 is shown in FIG. 24 and is provided for visible viewing of the inner mechanisms of restraint anchor device 501. View slot 506 is not provided on the commercially produced products, and it is provided in the embodiment shown only for the purpose of displaying the internal operation of the device.

Spanner key 598 can be gripped by the restraint anchor user, pushed straight downward to insert tub sealing element 530 into restraint anchor device 501 and then rotated clockwise (from a top perspective), to rotate tub sealing element 530 into final position. This is an improvement over the first alternative embodiment in that only two motions are needed to lock the tub sealing element 530. Further, the first motion is an easily excluded straight push downward of the tub sealing element 530 with engagement pins 543 sliding vertically through vertical slot portion 518″ of slot 518. This motion is relatively friction free and relies only on the user's physical strength, typically facilitated by the body weight and shoulder strength of the user. The closure motion is completed through the action of the engagement pins 543 moving horizontally through horizontal slot portion 518′. In the opening procedure, the action of the engagement pins 543 guided through the entire slot 518 causes the tub sealing element rise out of the tub 510 under the action of compressible element 526 (within the restraint anchor device 501).

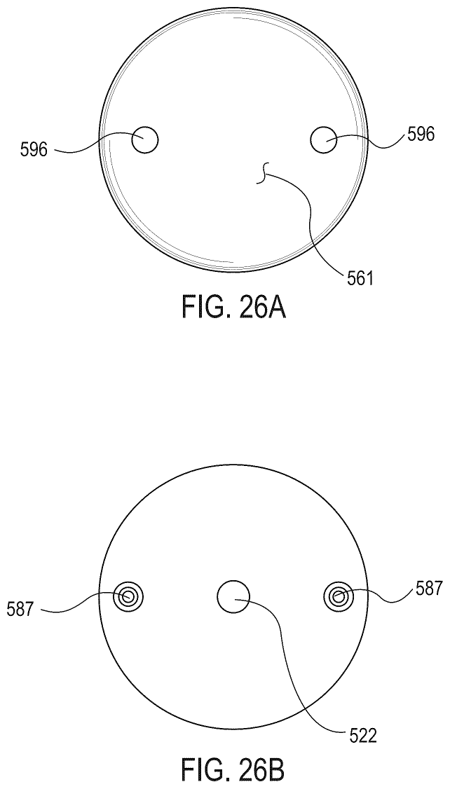

FIG. 26A and FIG. 26B show the top and bottom views, respectively, of the restraint anchor according to the second alternative embodiment. Notably different in this embodiment, the upper surfaces of the tub sealing element 530 is a single-piece, stainless steel cast element with a smooth and uninterrupted top surface, i.e. without any securing cap mechanism for locking. Also notably different, key holes 596 are provided through top restraint disk 562 and are replicated as a secondary set of aligned key holes 596′ through bottom restraint disk 566. Spanner key 598 includes longer engagement pins for insertion engagement through both sets of aligned holes an into the inner volume 520 of the tub 510.

FIG. 26B shows the bottom view of the restraint anchor according to a second alternative embodiment of the invention, including mounting hole 522, which may be threaded. Mounting hole 522 is used to securely mount the anti-ligature device within the environment as previously described.

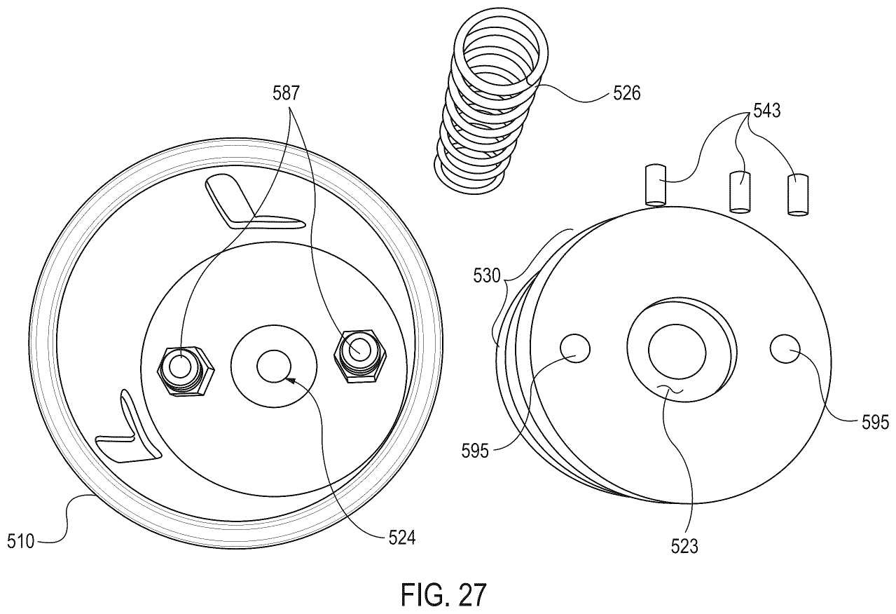

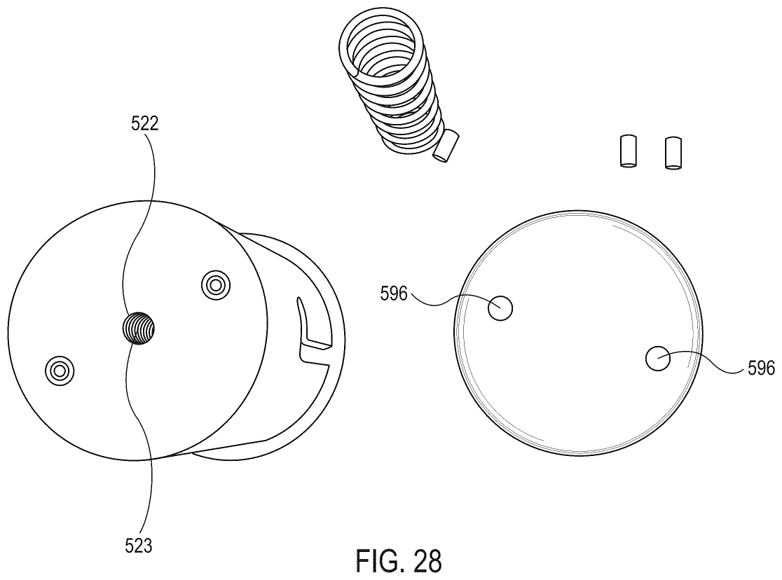

FIG. 27 shows a disassembly of the restraint anchor exposing the inner surfaces of the restraint anchor according to a second alternative embodiment of the invention. Included in that disassembly are top sealing element 530, tub 510, compressible element 526, and engagement pins 543 (unscrewed from top sealing element 530). Annular ring 524 is milled out of the inner surface 519 of tub bottom portion 516 to provide a seat for compressible element 526. Annular ring 523 is shown and is milled out of the bottom surface of bottom restraint disk 566 to provide the other seat for compressible element 526. Three slots 518 are provided around the perimeter of tub 510 for guiding the insertion of the tub sealing element 530 in the second embodiment. FIG. 28 shows a disassembly of the restraint anchor exposing the exterior surfaces of the restraint anchor according to the second alternative embodiment of the invention. FIG. 28 shows a closeup of the tub bottom and mounting hole 522 is shown with threads 523.

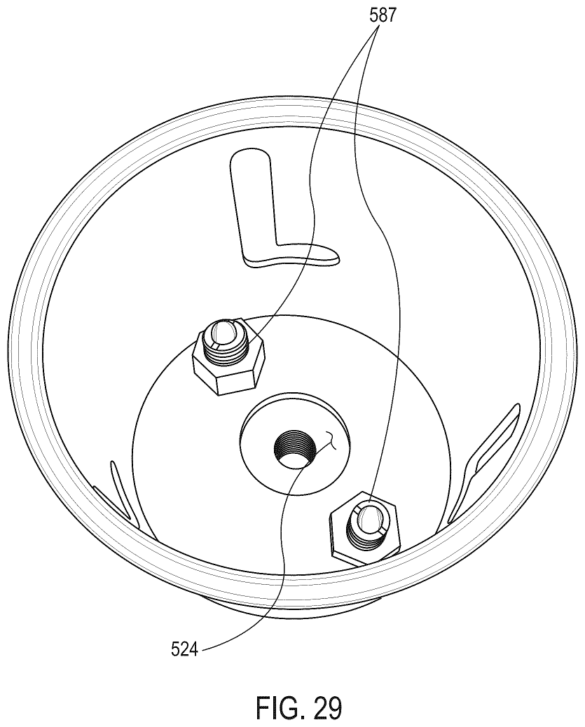

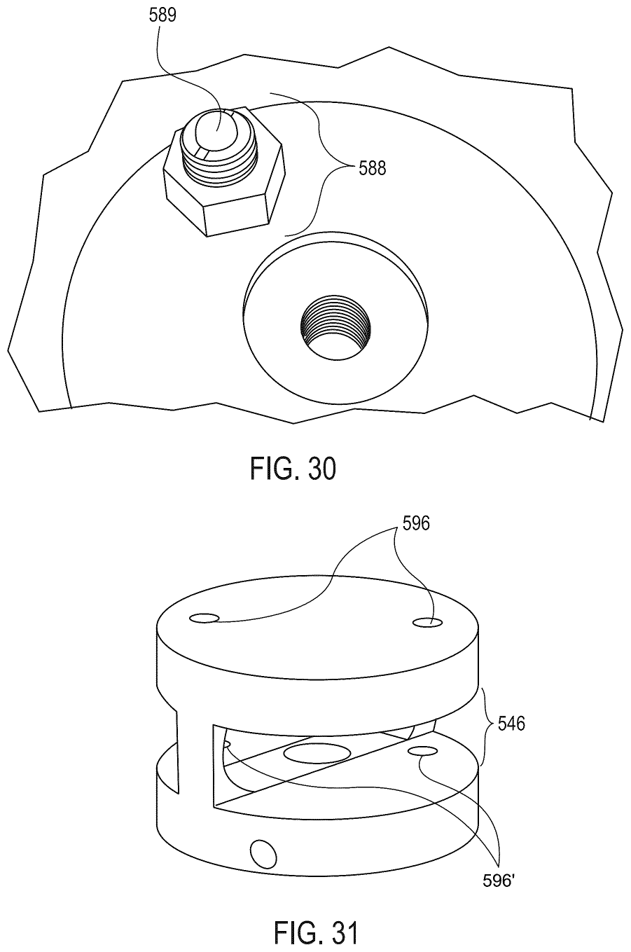

FIG. 29 and FIG. 30 show two closeups of the tub bottom portion 516 within the inner volume 520 according to the second alternative embodiment of the invention. As with the first alternative embodiment, a plunger 587 is provided for in the restraint anchor device. In the second embodiment that plunger 587 is disposed at bottom of tub 530. In FIG. 29, plunger 587 is shown as being threadably engaged with the tub 510. FIG. 28 shows the inserted side of plunger 587 as screwed into the tub 510 with the bottom surface of the insertable end of plunger 587 being coplanar with the bottom surface of the tub 510. Referring to FIG. 29 and FIG. 30, once inserted, the plunger extends up and into the inner volume 520 of tub 510 a certain distance 588. At the end of the plunger 587 is ball nose 589 that compresses into the plunger 587 when under force. Ball nose 589 is a metal ball that is situated within the plunger housing and is compressibly pushed within the plunger when the tub sealing element 530 is fully inserted into tub 510, and extends beyond the plunger tip when no pressure is applied to the ball nose 589.

One such plunger that may be used for this application is the McMaster-Carr Steel Long-Nose Spring Plunger. These plungers are made with differing thread count for insertion and different nose force application pressures to accommodate different force insertion design criteria.

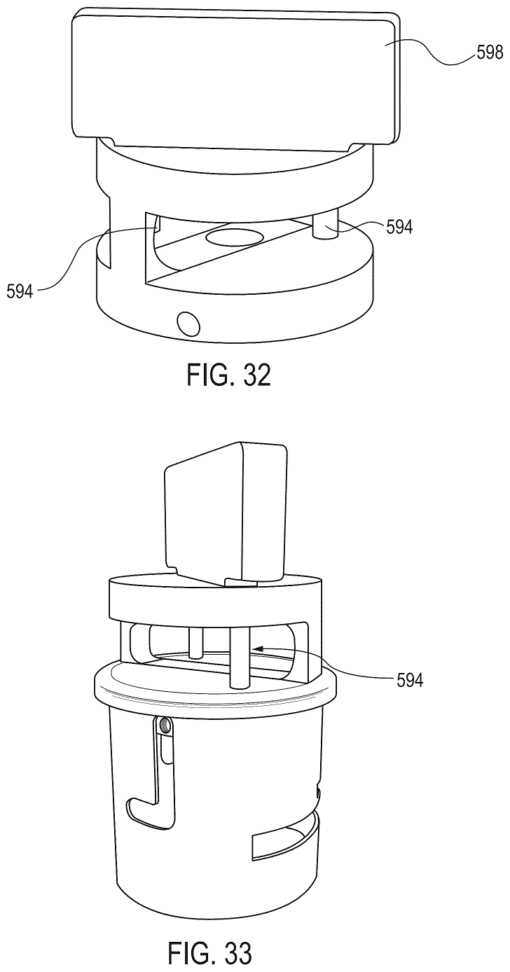

FIG. 31 and FIG. 32 show two oblique views of the tub sealing element 530 according to a second alternative embodiment of the invention. In FIG. 31, top key holes 596 and bottom key holes 596′ are clearly shown on opposite sides of the restraint slot 546. As shown in FIG. 32 and FIG. 33, these are in vertical alignment with each other so that spanner key pins 594 pass through both sets of key holes and into inner volume 520 of tub 510 when the spanner key is fully inserted through tub sealing element 530 and into tub 510.

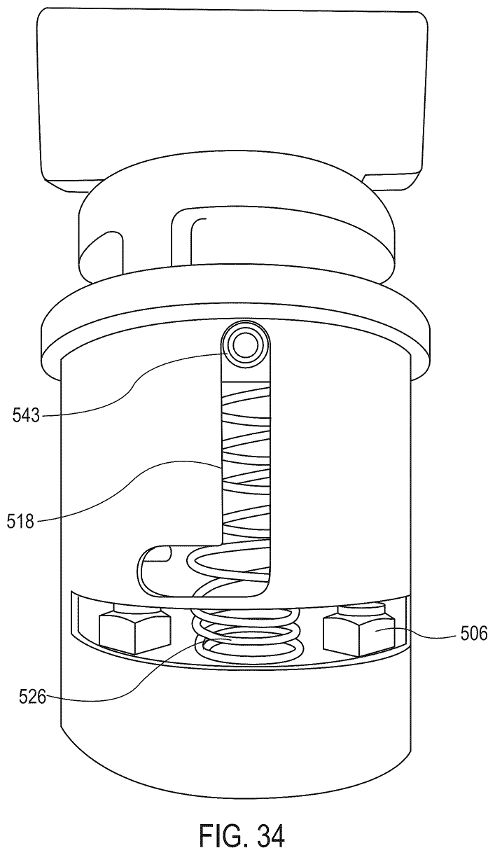

FIG. 34 shows a closeup side view of the restraint anchor device 510 with a clear view of compressible element 526 through slot 518 and view slot 506 with the restraint anchor in the open configuration and the spanner key fully inserted. There engagement pins 543 are threadably engaged with tub sealing element 530 and positioned within slots 518.

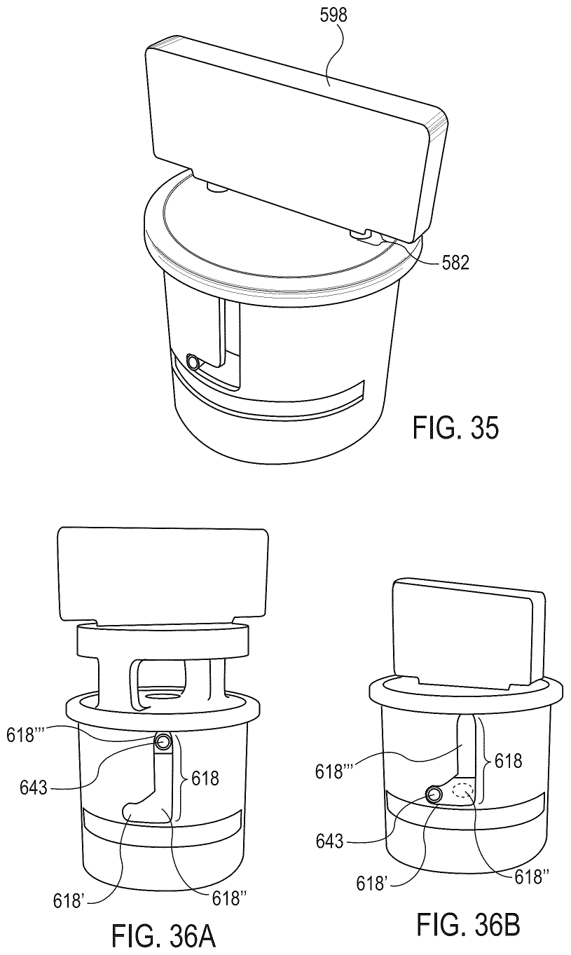

FIG. 35 shows an oblique view of the restraint anchor in a closed configuration according to the second alternative embodiment of the invention. As show, spanner key 598 is fully inserted into key holes 596 and 596′ and the bottom of the spanner key is disposed slightly above the top of tub sealing element 530 a distance 582. In this closed configuration, the spanner key pins 594 are sited directly on top of the ball noses 589 which are extended into the spanner key holes 596′ at the bottom surface of bottom restraint disk 566. As such a “locking mechanism” function is performed through the action of the ball noses 589 being extended and resting in the spanner key holes. When inserted in the closed configuration, the spanner key “bounces” a bit as the tips of the spanner key pins 594 push down on the ball noses 589.

FIGS. 36A and 36B shows a side view the tub sealing element 530 according to a third alternative embodiment of the invention. This embodiment is a slight variation of the second embodiment. In FIG. 36A the tub sealing element is shown inserted fully into the tub. In FIG. 36B, the tub sealing element is shown disposed outside the tub. In FIGS. 36A and 36B, the “L-shaped” slot is replaced with a more open side cutout area within slot 618 which provides for the same vertical slot portion 618′″, a shorter bottom, horizontal slot portion 618′ an open area 618″ joining the 618′″ and 618.′ The open area provides for an easier insertion of the tub sealing element. Further, the short bottom horizontal slot portion 618′ is only slightly larger then the diameter of the pin 643 such that there is only one lock position that is achieved by the restraint anchor and that is in the final and closed, locked position. This embodiment satisfies the safety requirements mandated by some hospital safety organizations, where only one locked position exists and no partially locked configurations exist for the restraint anchor.

First, it should be noted the parts involved in the overall invention are significantly reduced in both number and complexity. Second, the operation of the restraint anchor device is significantly simplified in the second alternative embodiment.

In operation, the patient restraint device 501 of the second alternative embodiment works as follows. In the open configuration, the ball noses 589 are in an extended positions, with no pressure being exerted on the ball noses. As the tub sealing element 530 is pushed into the tub 510, the engagement pins 543 guide the tub sealing element 530 straight down and into the tub 510 under the pressure of the compressible element 526. At the lowest point of travel along vertical slot 518″, the bottom surface of bottom restraint disk 566 pushes on and engages the ball noses 589. As the tub sealing element 530 begins to be rotated, the ball noses remain compressed throughout the rotation until the very end of the horizontal movement portion. The tub sealing element is moved to the end where it stops when engagement pins 543 reach the end of slot horizontal portion 518′. Using the second alternative embodiment, this may be done by operator hand pressure and without the use of the spanner key 598. If the spanner key is used, at the end of the closure motion, the spanner key pins 594 are pushed up a bit as the ball nose(s) 589 extend back out from plunger 587 a slight distance and insert themselves into the lower spanner key holes 596′ on the bottom surface of the bottom of bottom restraint disk portion 566. This achieves the locked position of the device in this second alternative embodiment. Further, as the tub sealing element 530 is rotated, the ball nose(s) 589, under compression, move along that bottom surface of bottom restraint disk portion 566 and the plunger(s) 587 and cause an additional upward pressure on the tub sealing element 530. This compression persists through to the end of the travel through horizontal slot portion 518′ and until ball nose(s) 589 are seated in spanner key holes 596′. As appreciated by those of skill in the art, the sizing of the plunger to the tub dimensions, combine with the rotational location of the plunger(s) within the tub and the position of the key holes on the tub sealing element are of critical importance for operational exactness. It is desirable that the inserted plunger bottom is smooth with the outside surface of tub bottom portion 516 and that the ball nose is compressed by only a certain amount at the point of maximum insertion of tub sealing element into the tub.

To unlock the restraint device, the spanner key 598 is inserted into holes 596 and 596′ and down into the inner volume of tub 530. The end tips of the spanner key pins 594 engage and push down on ball noses 589 to push them back into plunger 587 so as to free the locking of the tub sealing element by pushing the ball noses 589 out of holes 596′. This then allows for counter-clockwise rotation of the tub sealing element 530 and ultimately allowing the restraint device to achieve the open configuration under the action of compression element 526 as described above.

As an additional design feature, a spanner safety check key maybe designed such that the spanner pins 594 are shorter than the unlocking spanner key. Assuming that the length of those pins does not allow for ball noses 589 to be pushed down back into plunger 587 so as to free the locking of the tub sealing element, that safety spanner key can “test” the locking function of any give patient restraint device by simply twisting it once inserted into the tub sealing element 530. If the device is locked, the tub sealing element will not rotate. If the tub sealing element rotates, then relocking of the device can be performed to ensure that the device is in a safely locked position and not accessible by the patient.

This inner locking function using the plunger allows for two advantageous functions. First, the tub sealing element is not loose or free to slide circularly when in the closed configuration, but through the dual use of the lower spanner key holes 596′ and not through the use of separately milled divots. Second, as with the first alternative embodiment, the ball nose provides some upward pressure on the tub sealing element at maximal insertion the compression force of the compressible element may be reduced somewhat while the same upward force is maintained on the tub sealing element in the closed configuration. This in turn makes the closing function easier for the restraint anchor operator, i.e. less force is required to close the restraint anchor device. In fact, in empirical use, the amount of force needed to push the tub sealing element into the tub is facilitated to the point that a system user may employ only the palm the hand along with slight arm and shoulder pressure to complete the closure operation and without using the spanner key. This was not possible with previous embodiments. As mentioned, these additional safety features and ease-of-use enhancements are highly desirable in the complicated environment in which the restraint anchor operates.

The terms “a” or “an,” as used herein, are defined as one or more than one. The term “another,” as used herein, is defined as at least a second or more. The terms “including” and/or “having”, as used herein, are defined as comprising (i.e., open transition). The term “coupled” or “operatively coupled,” as used herein, is defined as connected, although not necessarily directly and mechanically.

While the invention has been disclosed in connection with the preferred embodiments shown and described in detail, various modifications and improvements thereon will become readily apparent to those skilled in the art. Accordingly, the spirit and scope of the present invention is not to be limited by the foregoing examples, but is to be understood in the broadest sense allowable by law.

Although various embodiments, which incorporate the teachings of the present invention have been shown and described in detail herein, those skilled in the art can readily devise many other varied embodiments that still incorporate these teachings.

The invention claimed is:

1. An anti-ligature device for use with restraints, said anti-ligature device comprising:
a tub, said tub being open at one end and having an inner surface, said tub inner surface partially defining an inner volume;
a tub sealing element, said tub sealing element having a restraint element for engaging the restraints, said tub sealing element being disposed apart from said tub in an open position of said anti-ligature device, said restraint element being exposed to the environment in said open position, the restraints capable of being operatively coupled to said restraint element in said open position; said tub sealing element having at least one engagement element for engaging said tub sealing element to said tub, said tub sealing element being inserted into said inner volume in a closed position of said anti-ligature device, said restraint element being disposed with said tub in said closed position, said tub sealing element making a seal with said open end of said tub in said closed position such that said restraint element is not exposed to the environment in said closed position and the restraints are not capable of being operatively coupled to said restraint element in said closed position.

2. The device of claim 1 wherein said engagement element is a slot said tub sealing element includes an engagement pin, said engagement pin disposed within said slot, said engagement pin within said slot guiding said movement of said tub sealing element between said open position and said closed position, said tub sealing element includes a compressible element on a lower surface of said tub sealing element, said compressible element extending into an indent on said inner surface of said tub when said device is in said closed position and locking said device in said closed position.

3. The device of claim 1 wherein said engagement element is a slot said tub sealing element includes an engagement pin, said engagement pin disposed within said slot, said engagement pin within said slot guiding said movement of said tub sealing element between said open position and said closed position, a compressible element on a lower surface of said tub, said compressible element extending into an indent on a lower surface of said tub sealing element when said device is in said closed position and locking said device in said closed position.

4. The device of claim 3 wherein said device is unlocked with a spanner key, said tub sealing element having holes on a surface exposed to the environment, said indents on said lower surface of said tub sealing element also being holes, said holes on said exposed surface and said holes on said lower surface being aligned, said spanner key \inserted into said holes on said exposed surface and said holes on said lower surface to disengage said compressible element when said device is in said closed position to unlock said device.

5. The device of claim 3 wherein said device is checked with a safety spanner key, said tub sealing element having holes on a surface exposed to the environment, said indents on said lower surface of said tub sealing element also being holes, said holes on said exposed surface, said safety spanner key inserted into said holes on said exposed surface and wherein said safety spanner key is incapable of contact with said compressible element when said device is in said closed position, said device remaining locked when said safety spanner key is inserted into said holes on said exposed surface.

6. The device of claim 3 wherein said slot includes a vertical portion and a horizontal portion, said engagement pin being disposed within said horizontal portion when said device is in said closed position.

7. The device of claim 6 wherein said horizontal portion is only slightly larger than said engagement pin such that there is only one locking position when said device is in said closed position.

8. The device of claim 3 further including a compressible element between said lower tub surface and said lower surface of said tub sealing element.

9. A method of locking an anti-ligature device, said anti-ligature device comprising a tub, said tub being open at one end and having an inner surface, said tub inner surface partially defining an inner volume, a tub sealing element, said tub sealing element having a restraint element, said tub sealing element being disposed apart from said tub in an open position of said anti-ligature device, said restraint element being exposed to the environment in said open position, the restraints capable of being operatively coupled to said restraint element in said open position, said tub sealing element being inserted into said inner cavity in a closed position of said anti-ligature device, said method comprising:
pushing said tub sealing element into said inner volume;
engaging a compressible element within said tub; and locking said tub sealing element in said closed position when said compressible element is disposed in an indent within said tub.

10. The method of claim 9 wherein said indent is a hole within a lower surface of said tub sealing element, said step of locking further including the step of inserting said compressible element into said hole.

11. The method of claim 9 further including sealing with said tub sealing element said open end of said tub in said closed position, said restraint element not being exposed to the environment in said closed position and the restraints are not capable of being operatively coupled to said restraint element in said closed position.

12. The method of claim 9 wherein said device includes a spanner key, said tub sealing element has holes on a surface exposed to the environment, said holes on said exposed surface and said holes on said lower surface being aligned, said method further comprising:
inserting said spanner key into said holes on said exposed surface and said holes on said lower surface to disengage said compressible element; and
unlocking said tub sealing element from said closed position.
